(19) United States
(12) Patent Application Publication
(10) Pub. No.: US 2004/0152929 A1
(43) Pub. Date: Aug. 5, 2004

(54) Process for vinyl chloride manufacture from ethane and ethylene with air feed and alternative hcl processing methods

(76) Inventors: William D Clarke, Brazoria, TX (US); Terry D Haymon, Brazoria, TX (US); John P Henley, Midland, MI (US); Daniel A Hickman, Midland, MI (US); Mark E Jones, Midland, MI (US); Matt C Miller, Lake Jackson, TX (US); Thomas E Morris, Lake Jackson, TX (US); Daniel J Reed, Angleton, TX (US); Lawrence J Samson, Lake Jackson, TX (US); Steven A Smith, Baton Rouge, LA (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/477,502

(22) PCT Filed: May 8, 2002
(86) PCT No.: PCT/US02/14801

(51) Int. Cl. C07C 17/152

(52) U.S. Cl. 570/224

(57) ABSTRACT

In one aspect, a process for producing vinyl chloride from ethane/ethylene involving: (a) combining ethane, ethylene, or mixtures thereof with an oxygen source and a chlorine source in a reactor containing a suitable catalyst under conditions sufficient to convert substantially all of the C2 hydrocarbon fed and to produce a product stream comprising vinyl chloride and hydrogen chloride; and (b) recycling unreacted hydrogen chloride back for use in Step (a). No C2 hydrocarbon recycle is required in this process. In another aspect, a process for producing vinyl chloride involving: (a) combining ethane, optionally ethylene, an oxygen source, and a chlorine source in a reactor containing a suitable catalyst under conditions sufficient to produce vinyl chloride and hydrogen chloride; (b) catalytically reacting said hydrogen chloride in a second reactor to provide a second reactor effluent essentially devoid of hydrogen chloride; and (c) recycling said second reactor effluent to step (a). Optionally, the second process may be run under conditions to react C2 hydrocarbons to extinction and thereby eliminate a need for C2 hydrocarbon recycle. Either process may include a hydrogenation unit for converting cis/trans-1,2-dichloroethylenes) to ethylene dichloride.

This invention is directed to an apparatus and process for producing vinyl chloride monomer from ethane and ethylene. Especially, this invention is directed to processes for producing vinyl chloride monomer (VCM) where (1) ethane concentration is significant in input streams to the affiliated reactor, and (2) consideration is given to alternative hydrogen chloride processing methods.

Vinyl chloride is a key material in modern commerce, and most processes deployed today derive vinyl chloride from 1,2-dichloroethane (EDC) where the EDC is first-derived from ethylene; so, at least a three-operation overall system is used (ethylene from primary hydrocarbons, preponderantly via thermal cracking; ethylene to EDC; and then EDC to vinyl chloride). There is an inherent long-felt need in the industry to move toward an approach where vinyl chloride is derived more directly and economically from primary hydrocarbons without a need to first manufacture and purify ethylene, and the inherent economic benefit related to this vision has inspired a significant amount of development.

As a first general area of development, ethane-to-vinyl manufacture is of interest to a number of firms engaged in vinyl chloride production, and a significant amount of literature on the subject is now available. The following paragraphs overview key work related to the embodiments presented in the new developments of the present disclosure.

GB Patent 1,039,369 entitled “CATALYTIC CONVERSION OF ETHANE TO VINYL CHLORIDE” which issued on Aug. 17, 1966 describes use of multivalent metals, including those in the lanthanum series, in the production of vinyl chloride from ethane. The patent describes use of certain catalysts provided that “steam, available chlorine and oxygen are used in specific controlled ratios.” The described system operates at a temperature of between 500 and 750° C. Available chlorine in the described technology optionally includes 1,2-dichloroethane.

GB Patent 1,492,945 entitled “PROCESS FOR PRODUCING VINYL CHLORIDE” which issued on Nov. 23, 1977 to John Lynn Barclay discloses a process for the production of vinyl chloride using lanthanum in a copper-based ethane-to-vinyl catalyst. The authors describe that the lanthanum is present to favorably alter the volatility of copper at the elevated temperature required for operation. Examples show the advantage of excess hydrogen chloride in the affiliated reaction.

GB Patent 2,095,242 entitled “PREPARATION OF MONOCHLORO-OLEFINS BY OXYCHLORINATION OF ALKANES” which issued on Sep. 29, 1982 to David Roger Pyke and Robert Reid describes a “process for the production of monochlorinated olefins which comprises bringing into reaction at elevated temperature a gaseous mixture comprising an alkane, a source of chlorine and molecular oxygen in the presence of a . . . catalyst comprising metallic silver and/or a compound thereof and one or more compounds of manganese, cobalt or nickel”. The authors indicate that mixtures of ethane and ethylene can be fed to the catalyst. No examples are given and the specific advantages of ethane/ethylene mixtures are not disclosed.

GB Patent 2,101,596 entitled “OXYCHLORINATION OF ALKANES TO MONOCHLORINATED OLEFINS” which issued on Jan. 19, 1983 to Robert Reid and David Pyke describes a “process for the production of monochlorinated olefins which comprises bringing into reaction at elevated temperature a gaseous mixture comprising an alkane, a source of chlorine and molecular oxygen in the presence of a . . . catalyst comprising compounds of copper, manganese and titanium and is useful in the production of vinyl chloride from ethane.” The authors further describe that “the products of reaction are, in one embodiment, isolated and used as such or are, in one embodiment, recycled . . . to the reactor . . . to increase the yield of monochlorinated olefin.” The authors indicate that mixtures of ethane and ethylene can be fed to the catalyst. No examples are given and the specific advantages of ethane/ethylene mixtures are not disclosed.

U.S. Pat. No. 3,629,354 entitled “HALOGENATED HYDROCARBONS” which issued on Dec. 21, 1971 to William Q. Beard, Jr. describes a process for the production of vinyl chloride and the co-production of ethylene from ethane in the presence of hydrogen chloride and oxygen. Preferred catalysts are supported copper or iron. An example in this patent shows excess hydrogen chloride (HCl) relative to ethane in the reaction. A ratio of one ethane to four hydrogen chlorides is used to produce a stream containing 38.4 percent ethylene (which requires no HCl to produce) and 27.9 percent vinyl chloride (which requires only one mole of HCl per mole of vinyl chloride to produce).

U.S. Pat. No. 3,658,933 entitled “ETHYLENE FROM ETHANE, HALOGEN AND HYDROGEN HALIDE THROUGH FLUIDIZED CATALYST” which issued on Apr. 25, 1972 to William Q. Beard, Jr. describes a process for production of vinyl halides in a three reactor system combining an oxydehydrogenation reactor, an oxyhalogenation reactor and a dehydrohalogenation reactor. The authors show that (oxy)halodehydrogenation of ethane is, in some cases, enhanced by addition of both halogen and hydrogen halide. As in U.S. Pat. No. 3,629,354, the ethylene generated produces VCM through conventional oxyhalogenation (oxychlorination) and cracking. HCl produced in the cracking operation is returned to the halodehydrogenation reactor.

U.S. Pat. No. 3,658,934 entitled “ETHYLENE FROM ETHANE AND HALOGEN THROUGH FLUIDIZED RARE EARTH CATALYST” which issued on Apr. 25, 1972 to William Q. Beard, Jr. and U.S. Pat. No. 3,702,311 entitled “HALODEHYDROGENATION CATALYST” which issued on Nov. 7, 1972 to William Q. Beard, Jr. both describe a process for production of vinyl halides in a three reactor system combining a halodehydrogenation reactor, an oxyhalogenation reactor and a dehydrohalogenation reactor. The authors describe the halodehydrogenation of ethane to produce ethylene for subsequent conversion to EDC through oxyhalogenation (oxychlorination) with subsequent production of VCM through conventional thermal cracking. HCl produced in the cracking operation is returned to the oxyhalogenation reactor in '934 and to the halodehydrogenation reactor in '311. In the latter patent, the advantages of excess total chlorine, as both HCl and Cl₂ are shown to augment yield of desirable products.

U.S. Pat. No. 3,644,561 entitled “OXYDEHYDROGENATION OF ETHANE” which issued on Feb. 22, 1972 to William Q. Beard, Jr. and U.S. Pat. No. 3,769,362 entitled “OXYDEHYDROGENATION OF ETHANE” which issued on Oct. 30, 1973 to William Q. Beard, Jr. relate closely to those above and describe processes for the oxydehydrogenation of ethane to ethylene in the presence of excess quantities of hydrogen halide. The patent describes a catalyst of either copper or iron halide further stabilized with rare earth halide where the ratio of rare earth to copper or iron halide is greater than 1:1. The patent describes use of a substantial excess of HCl relative to the molar amount of ethane fed, the HCl being unconsumed in the reaction.

U.S. Pat. No. 4,046,823 entitled “PROCESS FOR PRODUCING 1,2-DICHLOROETHANE” which issued on Sep. 6, 1977 to Ronnie D. Gordon and Charles M. Starks describes a process for the production of EDC where ethane and chlorine are reacted in the gas-phase over a copper containing catalyst.

U.S. Pat. No. 4,100,211 entitled “PROCESS FOR PREPARATION OF ETHYLENE AND VINYL CHLORIDE FROM ETHANE” which issued on Jul. 11, 1978 to Angelo Joseph Magistro describes regeneration of an iron catalyst for a process which reacts ethane into both ethylene and VCM in a mixture. This patent describes that a chlorine source is present from 0.1 mole to 10 moles per mole of ethane. In general, as the ratio of hydrogen chloride to ethane is increased, the yield of vinyl chloride and other chlorinated products also increases even as the yield of ethylene decreases.

U.S. Pat. No. 4,300,005 entitled “PREPARATION OF VINYL CHLORIDE” which issued on Nov. 10, 1981 to Tao P. Li suggests a copper-based catalyst for production of VCM in the presence of excess HCl.

U.S. Pat. No. 5,097,083 entitled “PROCESS FOR THE CHLORINATION OF ETHANE” which issued on Mar. 17, 1992 to John E. Stauffer describes chlorocarbons as a chlorine source in an ethane-to-VCM process. This patent describes methods where chlorohydrocarbons may be used to capture HCl for subsequent use in the production of vinyl.

EVC Corporation has been active in ethane-to-vinyl technology, and the following four patents have resulted from their efforts in development.

EP 667,845 entitled “OXYCHLORINATION CATALYST” which issued on Jan. 14, 1998 to Ray Hardman and Ian Michael Clegg describes a copper-based catalyst with a stabilization package for ethane-to-vinyl catalysis. This catalyst appears to be relevant to the further technology described in the following three US patents.

U.S. Pat. No. 5,663,465 entitled “BY-PRODUCT RECYCLING IN OXYCHLORINATION PROCESS” which issued on Sep. 2, 1997 to Ian Michael Clegg and Ray Hardman describes a method for the catalytic conversion of ethane to VCM which combines ethane and a chlorine source in an oxychlorination reactor with a suitable catalyst; recycles the byproducts to the oxychlorination reactor; treats unsaturated chlorinated hydrocarbon byproducts in a hydrogenation step to convert them to their saturated counterparts and passes them back to the reactor; and chlorinates ethylene byproduct to 1,2-dichloroethane for recycle.

U.S. Pat. No. 5,728,905 entitled “VINYL CHLORIDE PRODUCTION PROCESS” which issued on Mar. 17, 1998 to Ian Michael Clegg and Ray Hardman discusses ethane-to-vinyl manufacture in the presence of excess HCl using a copper catalyst. The patent describes a process of catalytic oxychlorination of ethane between ethane, an oxygen source and a chlorine source in the presence of a copper and alkali metal-containing catalyst. HCl is supplied to the oxychlorination reactor in excess of the stoichiometric requirement for chlorine.

U.S. Pat. No. 5,763,710 entitled “OXYCHLORINATION PROCESS” which issued on Jun. 9, 1998 to Ian Michael Clegg and Ray Hardman discusses catalytic oxychlorination of ethane to VCM by combining ethane and a chlorine source in an oxychlorination reactor in the presence of an oxychlorination catalyst (the reaction conditions selected to maintain an excess of HCl); separating the VCM products; and recycling by-products to the reactor.

Turning now to art in the derivation of vinyl chloride from ethylene, most commercial processes for the production of VCM use ethylene and chlorine as key raw materials. Ethylene is contacted with chlorine in liquid 1,2-dichloroethane containing a catalyst in a direct chlorination reactor. The 1,2-dichloroethane is subsequently cracked at elevated temperature to yield VCM and hydrogen chloride (HCl). The HCl produced is in turn fed to an oxychlorination reactor where it is reacted with ethylene and oxygen to yield more 1,2-dichloroethane. This 1,2-dichloroethane is also fed to thermal cracking to produce VCM. Such a process is described in U.S. Pat. No. 5,210,358 entitled “CATALYST COMPOSITION AND PROCESS FOR THE PREPARATION OF ETHYLENE FROM ETHANE” which issued on May 11, 1993 to Angelo J. Magistro.

The three unit operations (direct chlorination, oxychlorination and thermal cracking) of most presently used commercial processes are frequently referenced in combination as a “balanced” EDC plant, although additional sources of chlorine (HCl) are, in one embodiment, also brought into these extended plant systems. The net stoichiometry of the “balanced” plant is:

4C₂H₄+2Cl₂+O₂→4C₂H₃Cl+2H₂O

Ethylene cost represents a significant fraction of the total cost of production of VCM and requires expensive assets to produce. Ethane is less expensive than ethylene, and production of VCM from ethane should, therefore, reasonably lower the production cost of VCM in comparison to the production cost of VCM when manufactured primarily from purified and separated ethylene.

It is common to refer to the conversion of ethylene, oxygen and hydrogen chloride to 1,2-dichloroethane as oxychlorination. Catalysts for the production of 1,2-dichloroethane by oxychlorination of ethylene share many common characteristics. Catalysts capable of performing this chemistry have been classified as modified Deacon catalysts [Olah, G. A., Molnar, A., Hydrocarbon Chemistry, John Wiley & Sons (New York, 1995), pg 226]. Deacon chemistry refers to the Deacon reaction, the oxidation of HCl to yield elemental chlorine and water. Other authors have offered that oxychlorination is the utilization of HCl for chlorination and that the HCl is converted oxidatively into Cl₂ by means of the Deacon process [Selective Oxychlorination of Hydrocarbons: A Critical Analysis, Catalytica Associates, Inc., Study 4164A, October 1982, page 1]. The ability of oxychlorination catalysts to produce free chlorine (Cl₂) thus defines them. Indeed, oxychlorination of alkanes has been linked to the production of free chlorine in the system [Selective Oxychlorination of Hydrocarbons: A Critical Analysis, Catalytica Associates, Inc., Study 4164A, October 1982, page 21 and references therein]. These catalysts employ supported metals capable of accessing more than one stable oxidation state, such as copper and iron. In the conventional technology, oxychlorination is the oxidative addition of two chlorine atoms to ethylene from HCl or another reduced chlorine source.

Production of vinyl from ethane can proceed via oxychlorination provided catalysts are present which are capable of production of free chlorine. Such catalysts will convert ethylene to 1,2-dichloroethane at low temperatures. At higher temperatures, 1,2-dichloroethane will be disposed to thermally crack to yield HCl and vinyl chloride. Oxychlorination catalysts chlorinate olefinic materials to still higher chlorocarbons. Thus, just as ethylene is converted to 1,2-dichloroethane, vinyl chloride is converted to 1,1,2-trichloroethane. Processes using oxychlorination catalysts inherently produce higher chlorinated side-products. This is examined in patents to EVC (EP 667,845, U.S. Pat. No. 5,663,465, U.S. Pat. No. 5,728,905, and U.S. Pat. No. 5,763,710), which show high levels of multichlorinated side-products being produced over the oxychlorination catalyst used. In consideration of the above, a number of concepts regarding the use of ethane to produce VCM have clearly been described previously. Catalysts employed most frequently are modified Deacon catalysts operated at sufficiently higher temperatures (>400° C.) than those required to perform ethylene oxychlorination (<275° C.). Catalysts used for ethane-to-VCM manufacture are frequently stabilized against the migration of the first-row transition metals, as described and reviewed in GB Patent 1,492,945; GB Patent 2,101,596; U.S. Pat. No. 3,644,561; U.S. Pat. No. 4,300,005; and U.S. Pat. No. 5,728,905.

Use of chlorocarbons as chlorine sources in ethane-to-VCM processes has been disclosed in GB Patent 1,039,369; GB Patent 2,101,596; U.S. Pat. No. 5,097,083; U.S. Pat. No. 5,663,465; and U.S. Pat. No. 5,763,710. GB Patent 1,039,369 requires that water be fed to the reactor system. GB Patent 2,101,596 is specific to copper catalysts. U.S. Pat. No. 5,663,465 describes a process which uses a direct chlorination step to convert ethylene to EDC prior to feeding it back to the VCM reactor.

Notwithstanding a relatively qualitative reference in GB Patent 2,095,242, another recent development in ethylene-to-vinyl processes is outlined in Dow Case No. 44649 to Mark E. Jones, Michael M. Olken, and Daniel A. Hickman, entitled “A PROCESS FOR THE CONVERSION OF ETHYLENE TO VINYL CHLORIDE, AND NOVEL CATALYST COMPOSITIONS USEFUL FOR SUCH PROCESS”, filed as International Patent Application Serial No. PCT/US00/27272, on Oct. 3, 2000, published on May 31, 2001, as International Patent Publication no. WO 01/38273. The catalyst of this application demonstrates utility in reacting significant quantities of both ethane and ethylene into vinyl chloride monomer and thereby opens a door to new approaches in processes for vinyl chloride manufacture. However, the catalyst action yields hydrogen chloride in the reaction product. In this regard, management of hydrogen chloride (and affiliated hydrochloric acid) within the process is a key issue to be resolved when a catalyst system capable of conversion of both ethane and ethylene into vinyl chloride monomer is used. In contemplation of vinyl chloride facility construction, there is also a need to enable use of prior equipment as much as possible, where some existing equipment may have the ability to handle hydrogen chloride and other existing equipment does not have the ability to handle hydrogen chloride. The present invention provides embodiments for fulfilling these needs, by providing an apparatus and process for handling hydrogen chloride generated from the ethane/ethylene-to-vinyl reactor by essentially fully recovering it from the reactor effluent in the first unit operation after the ethane/ethylene-to-vinyl reaction step or stage.

In one aspect, this invention provides a method of manufacturing vinyl chloride, comprising: (a) combining reactants including ethane, ethylene, or mixtures thereof with an oxygen source and a chlorine source in a reactor containing a suitable catalyst under conditions sufficient to convert substantially all of the C2 hydrocarbon fed and to produce a product stream comprising vinyl chloride and hydrogen chloride; and (b) recycling unreacted hydrogen chloride back for use in the combining step. This process can be run using air as an oxygen source. Additional features and advantages of the present invention are more fully apparent from a reading of the detailed description of the invention and the drawings.

In another aspect, this invention provides a method of manufacturing vinyl chloride, comprising: (a) combining reactants including ethane and optionally ethylene with an oxygen source and a chlorine source in a reactor containing a suitable catalyst under conditions sufficient to produce vinyl chloride and hydrogen chloride; (b) catalytically reacting essentially all of said hydrogen chloride in a second reactor to provide a second reactor effluent essentially devoid of hydrogen chloride; and (c) recycling said second reactor effluent to catalytically react together with said ethane, said optional ethylene, said oxygen source, and said chlorine source in said combining step. Additional features and advantages of the second aspect of the invention are more fully apparent from a reading of the detailed description of the invention and the drawings.

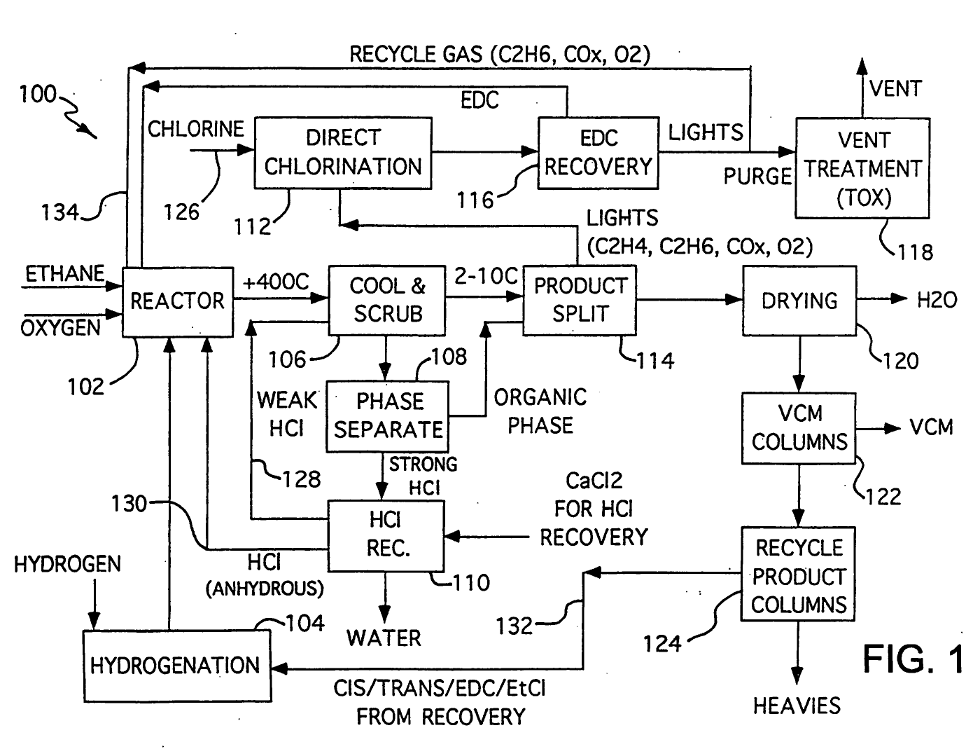

FIG. 1 shows characterization, as best understood from earlier publications, of a contemplated ethane-to-vinyl chloride process employing a catalyst capable of converting ethane to VCM.

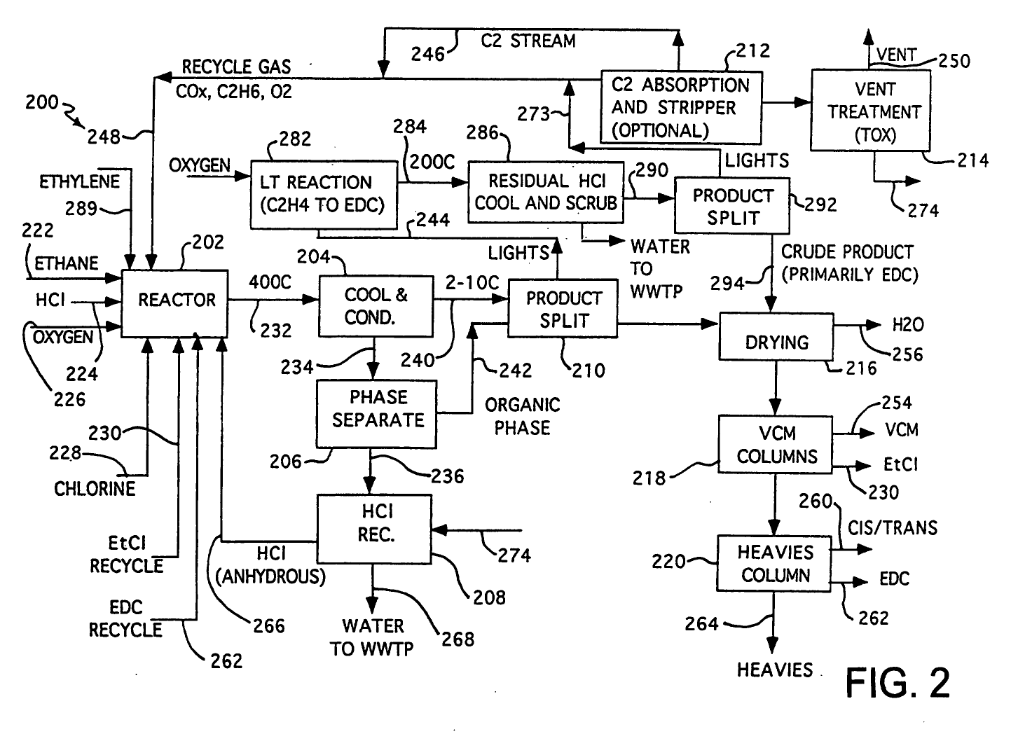

FIG. 2 shows an ethane/ethylene-to-vinyl chloride process, as reproduced from FIG. 2 of International Patent Publication No. WO 01/38272 (May 31, 2001). The process employs a catalyst capable of converting ethane and ethylene to VCM via oxydehydro-chlorination with a second oxychlorination reactor for ethylene conversion to ethylene dichloride.

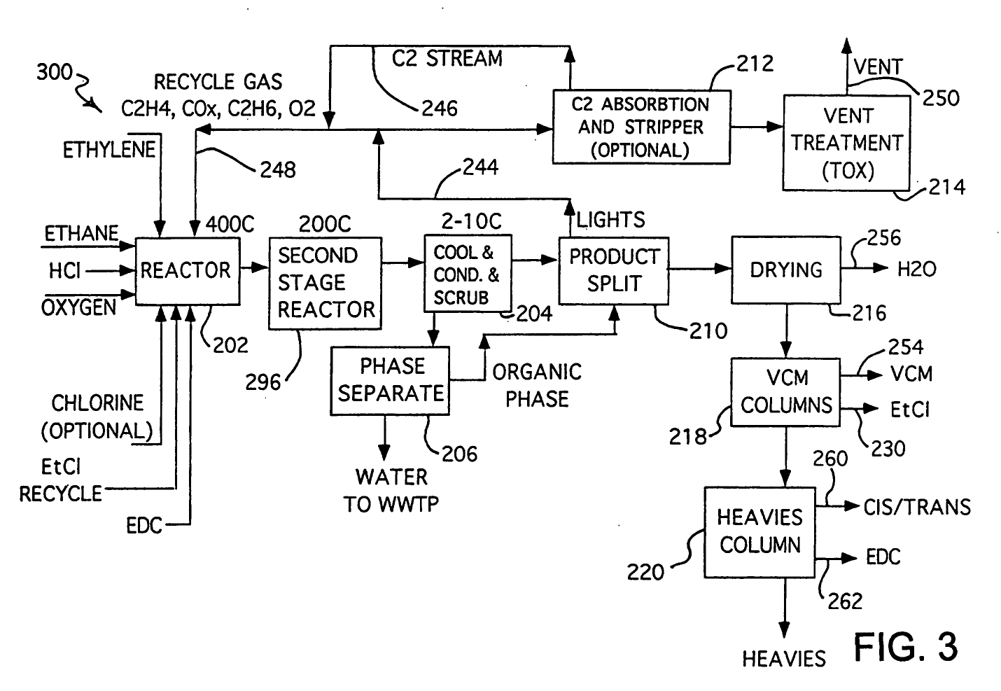

FIG. 3 shows an ethane/ethylene-to-vinyl chloride process, as reproduced from FIG. 3 of International Patent Publication No. WO 01/38272 (May 31, 2001). The process employs a catalyst capable of converting ethane and ethylene to VCM via oxydehydro-chlorination in a first reactor with a second stage reactor system.

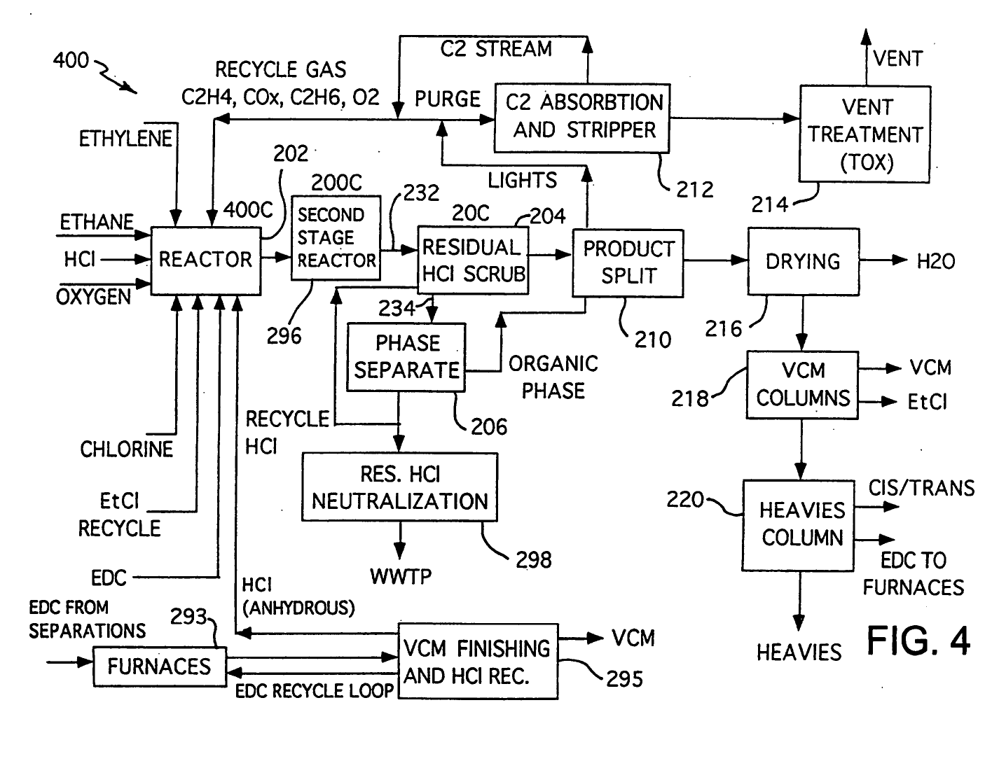

FIG. 4, reproduced from FIG. 4 of International Patent Publication No. WO 01/38272 (May 31, 2001), shows the ethane/ethylene-to-vinyl chloride process of FIG. 3 with an incorporated vinyl furnace and vinyl finishing operation.

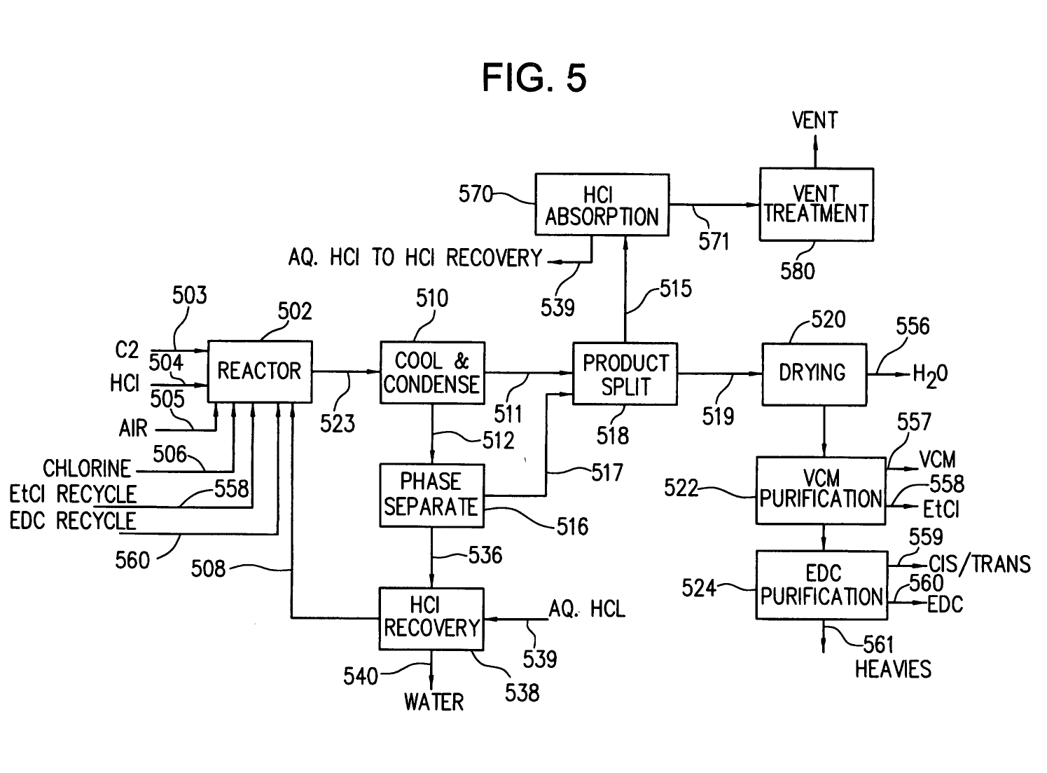

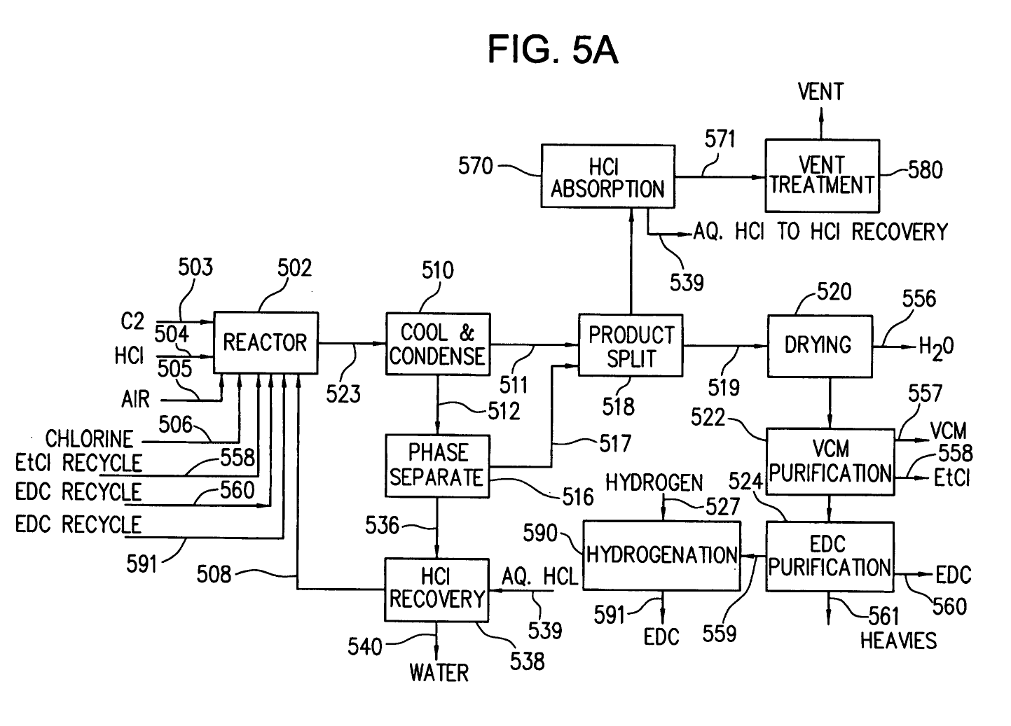

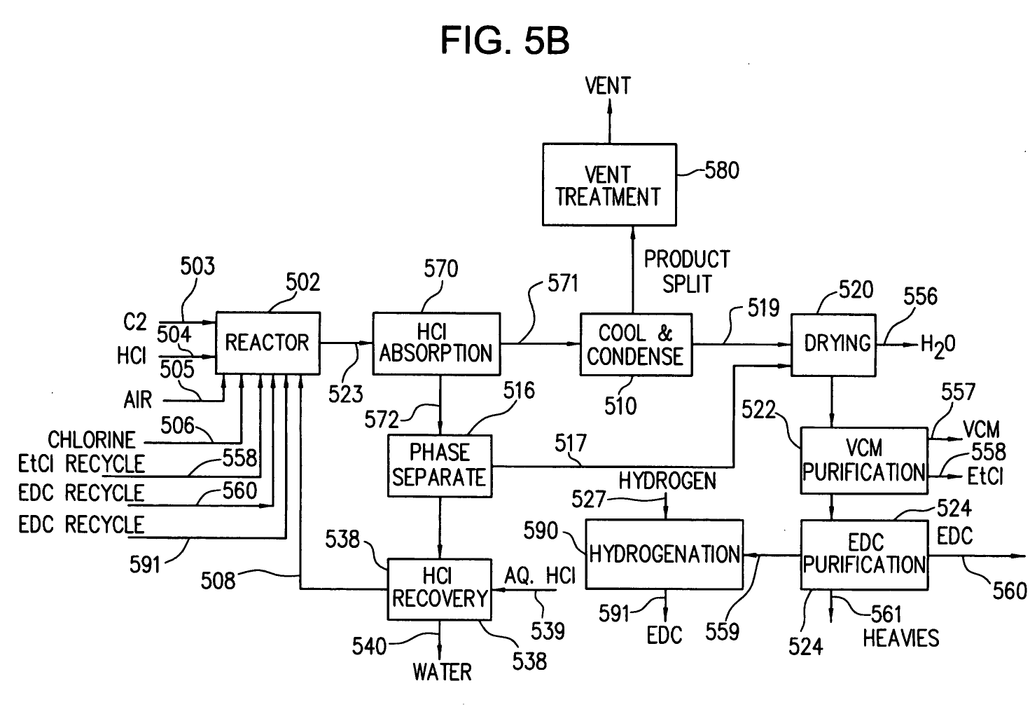

FIG. 5 illustrates an embodiment of this invention wherein air is employed as the source of oxygen for the ethane/ethylene-to-vinyl chloride process with hydrogen chloride recovery and recycle. In this embodiment, C2 hydrocarbon starting materials are reacted essentially to extinction, thereby eliminating a C2 hydrocarbon recycle.

FIG. 5a illustrates another embodiment of this invention employing air as an oxygen source, similar to the embodiment of FIG. 5, with the exception that a hydrogenation unit operation is added to convert cis/trans-dichloroethylenes to 1,2-dichloroethane (ethylene dichloride, EDC).

FIG. 5b illustrates another embodiment of this invention employing air as an oxygen source, similar to the embodiment of FIG. 5, with the exception that FIG. 5b has a different configuration of unit operations from FIGS. 5 and 5a.

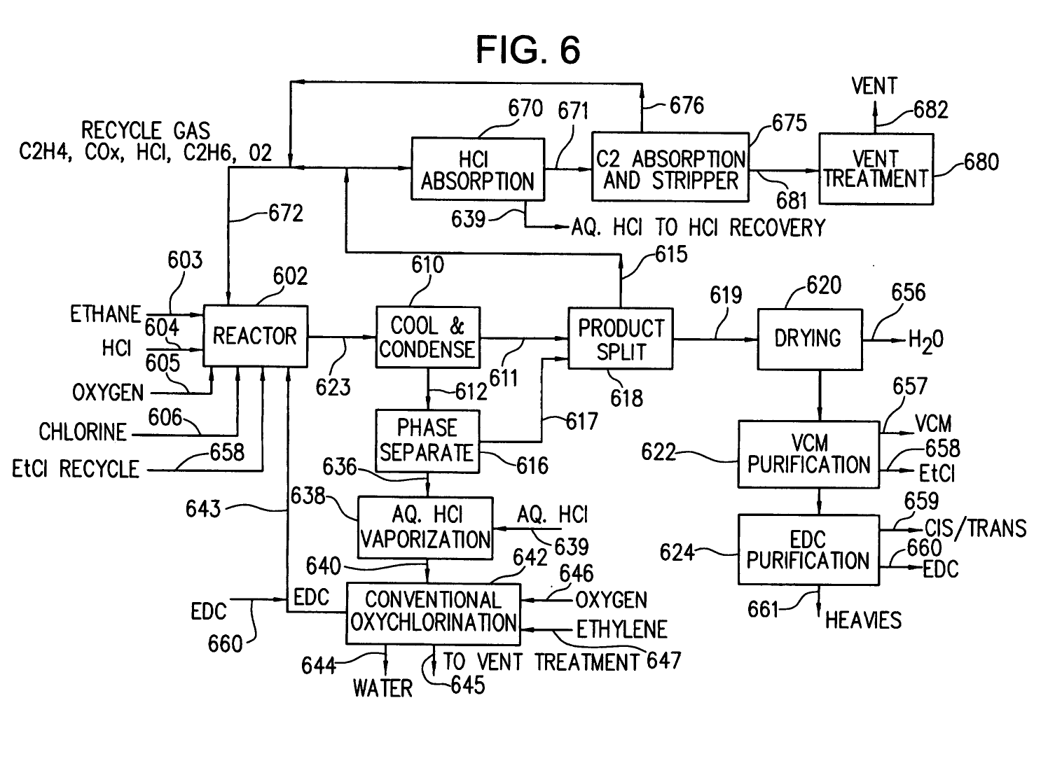

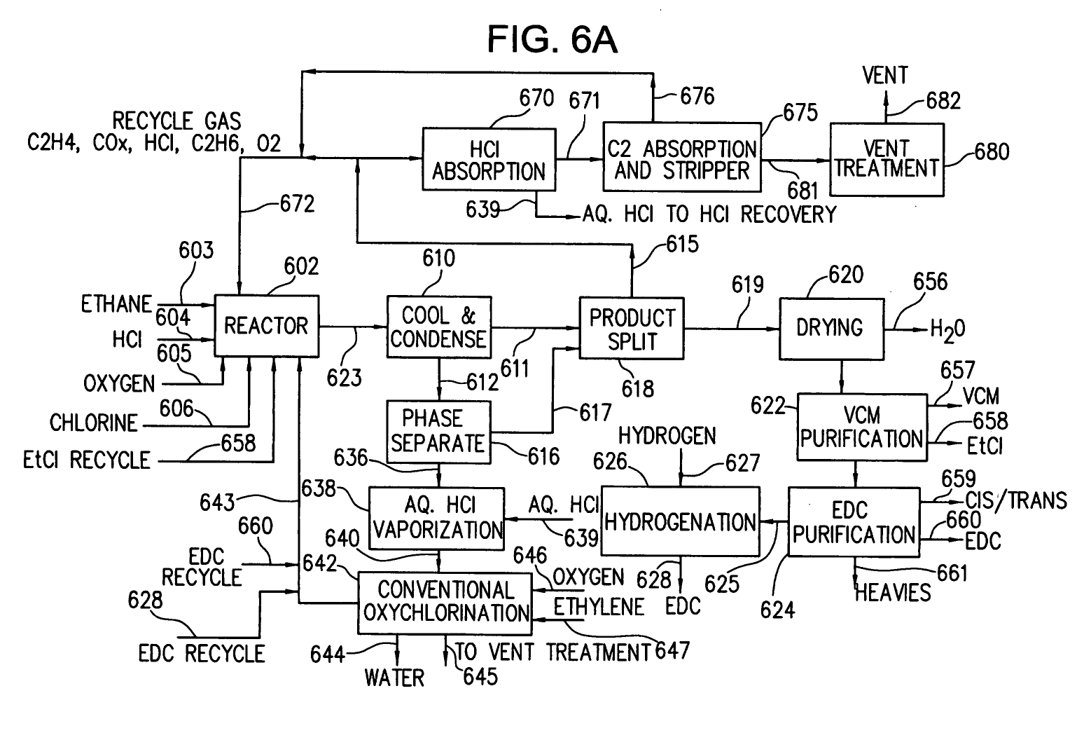

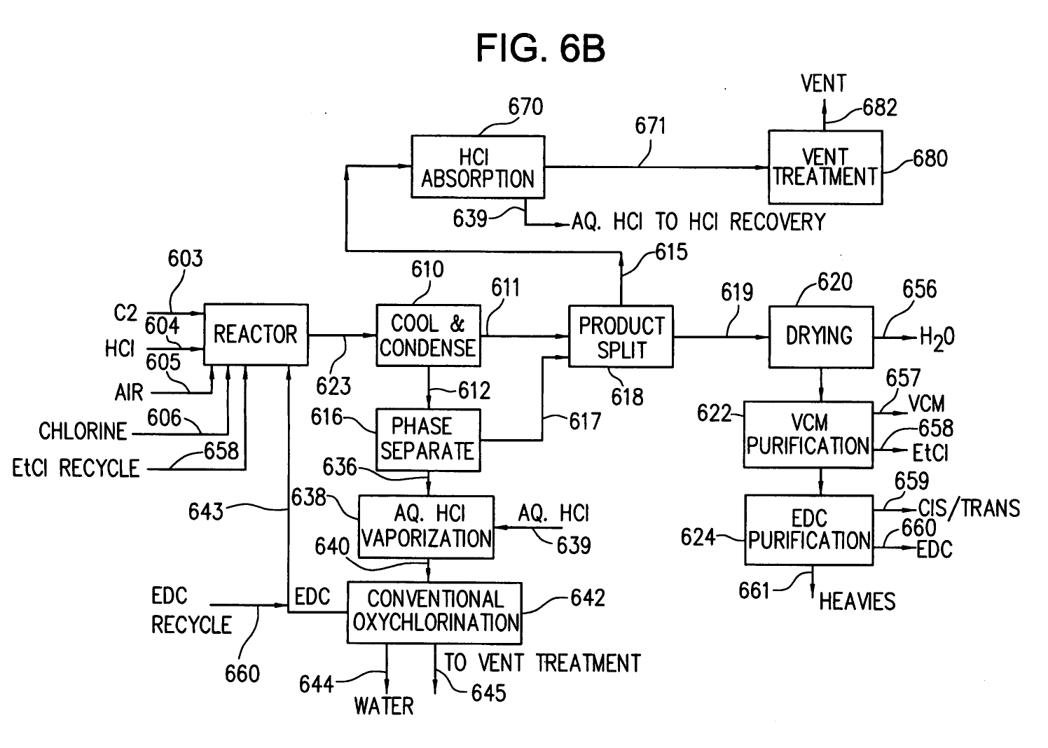

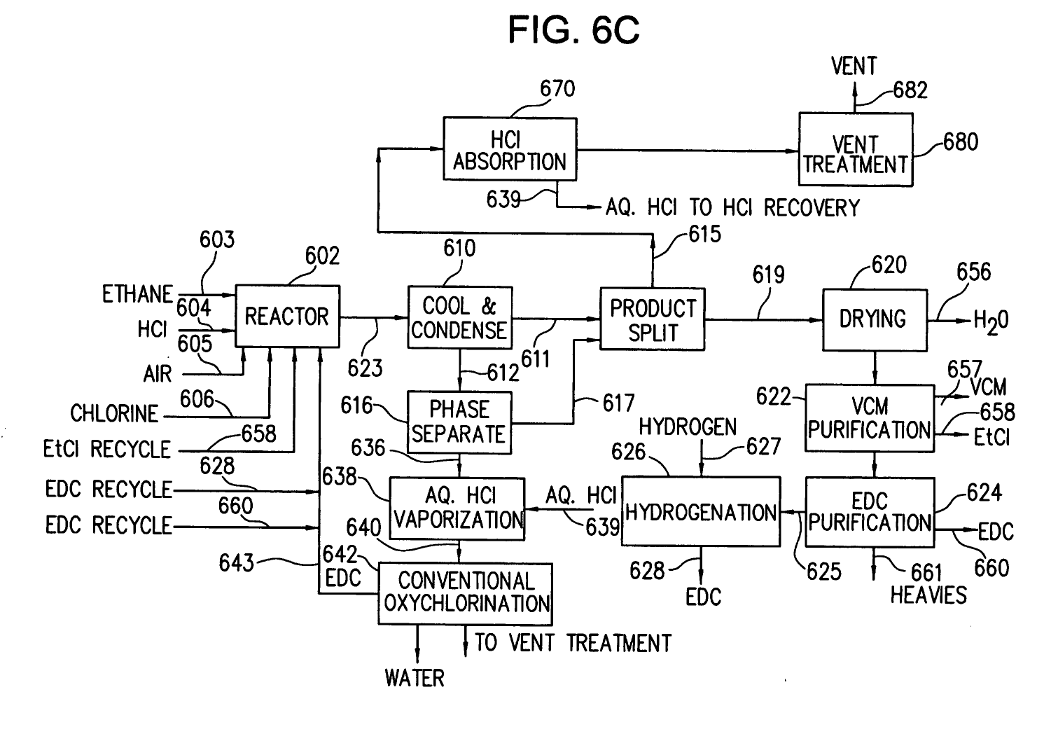

FIG. 6 illustrates an embodiment of this invention wherein HCl from the ethane/ethylene-to-vinyl chloride process is employed as a “wet” feed to a conventional oxychlorination reactor in which oxygen and ethylene are fed to form ethylene dichloride, which is recycled back to the primary reactor.

FIG. 6a illustrates an alternative scheme to the embodiment of FIG. 6, wherein a hydrogenation step is included for hydrogenating cis,trans-1,2-dichloroethlenes to 1,2-dichloroethane.

FIG. 6b illustrates another alternative scheme to the embodiment of FIG. 6, wherein a C2 absorption and stripper block and recycle is omitted since the C2 hydrocarbon starting materials are reacted essentially to extinction.

FIG. 6c illustrates another alternative scheme to the embodiment of FIG. 6 wherein the C2 absorption and stripper block and recycle is omitted and a hydrogenation unit is included.

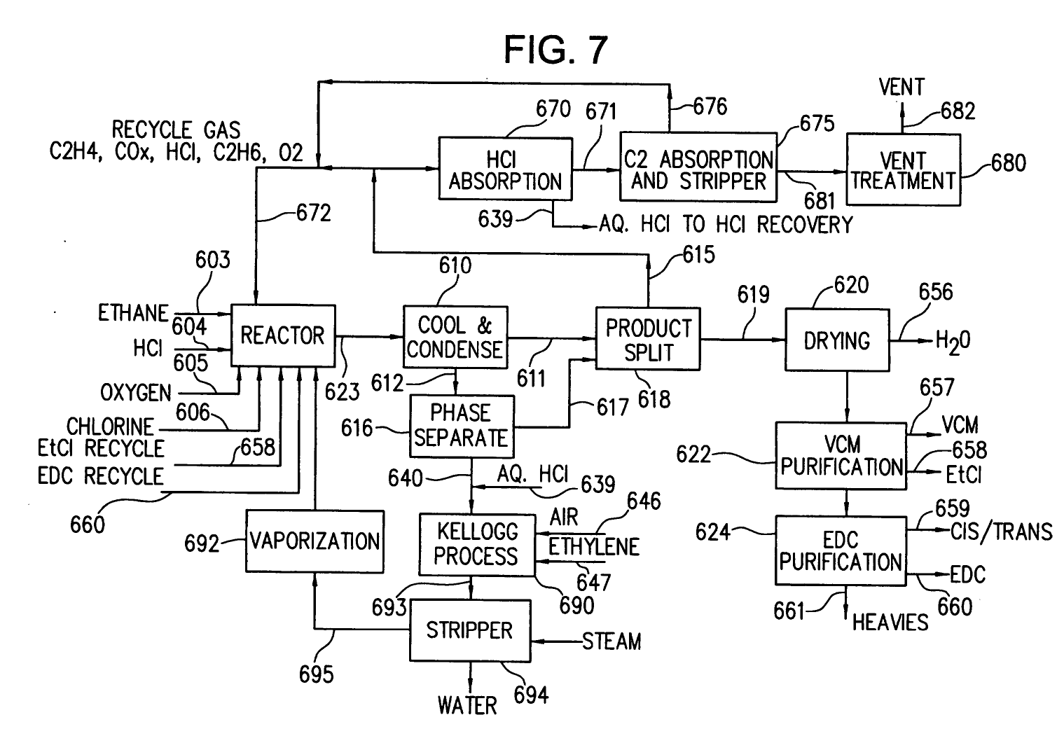

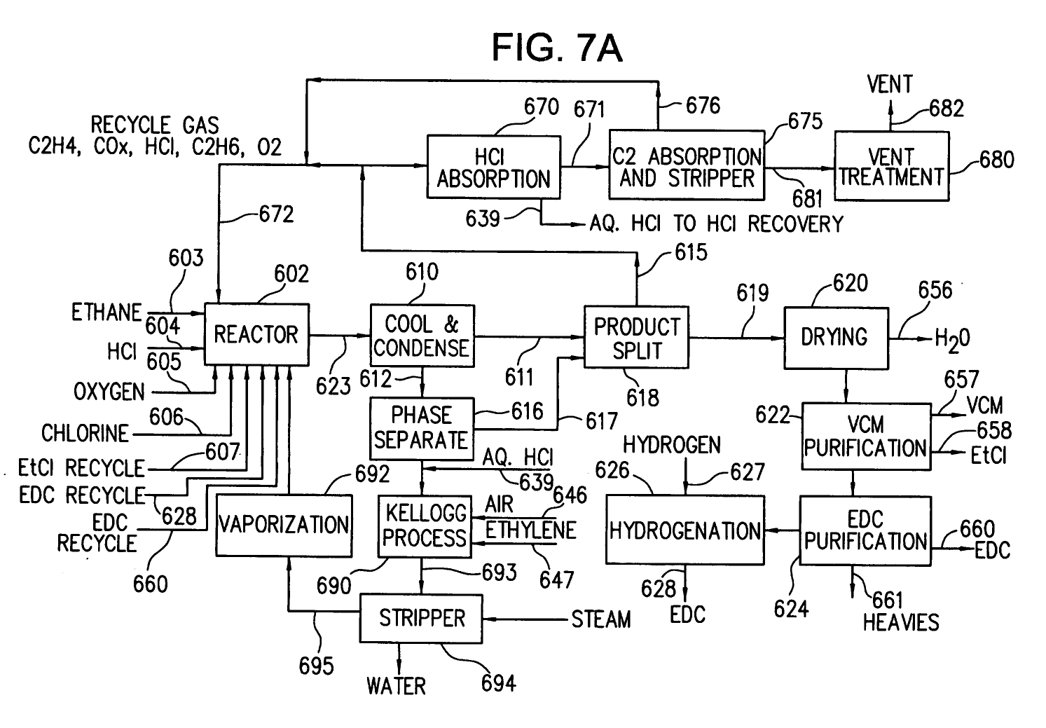

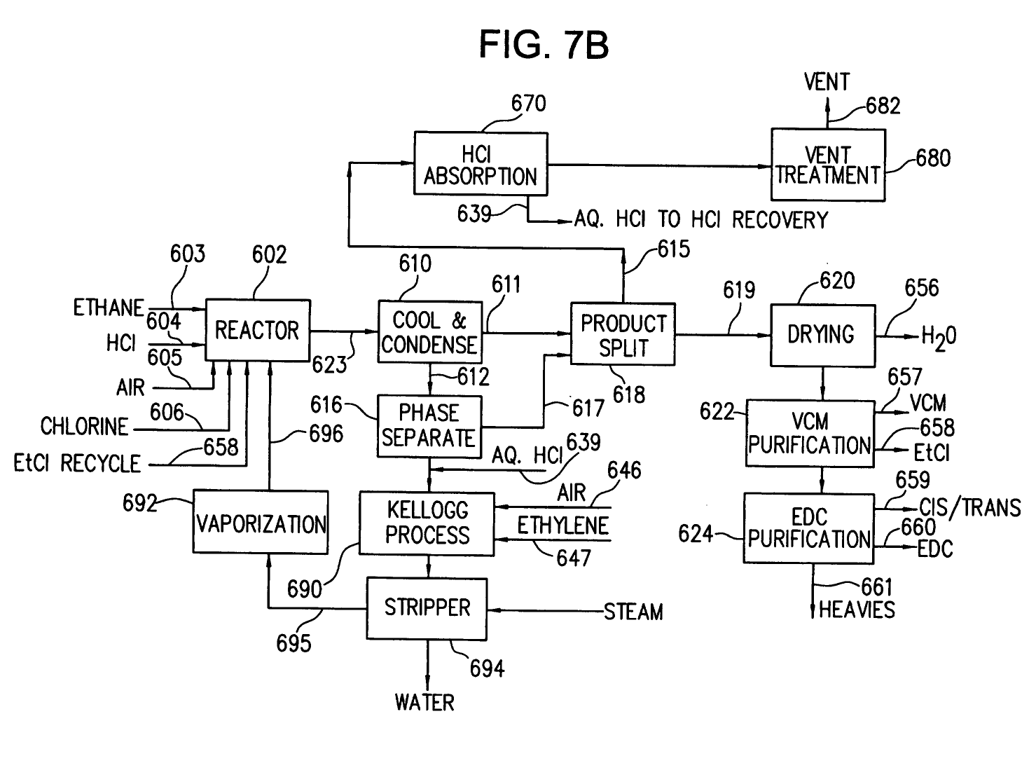

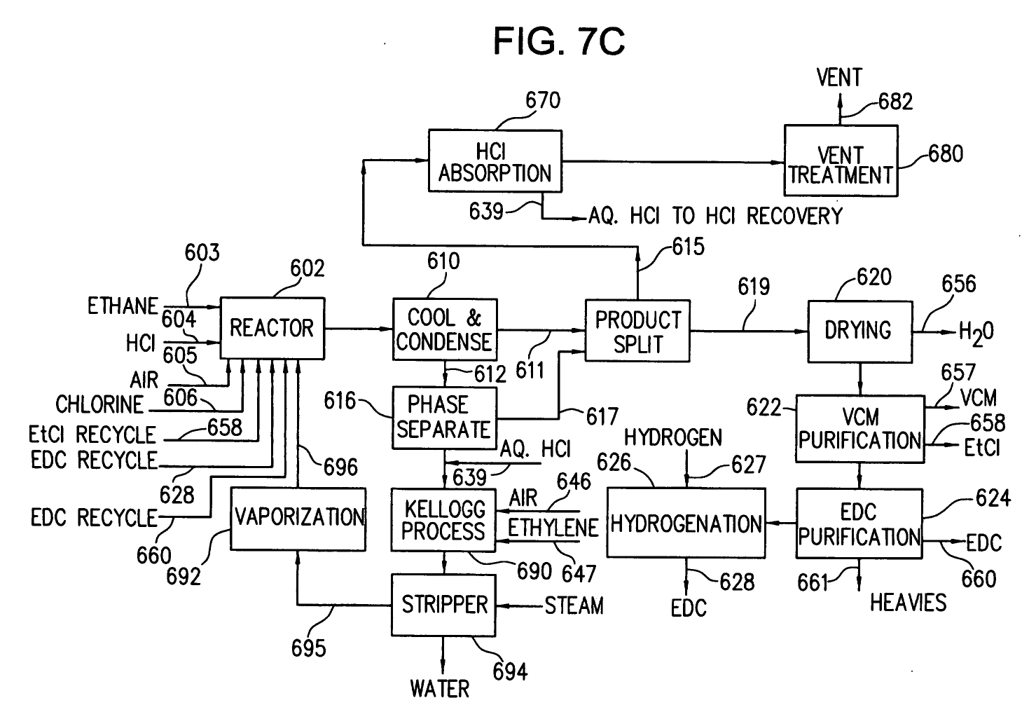

FIG. 7 illustrates another alternative scheme to the embodiment of FIG. 6, wherein aqueous HCl (the HCl is formed in the primary reactor) is used to form ethylene chloride and ethylene dichloride, which are fed to the primary reactor.

FIG. 7a illustrates an alternative scheme to the embodiment of FIG. 7 wherein a hydrogenation step is included.

FIG. 7b illustrates an alternative scheme to the embodiment of FIG. 7 wherein a hydrogenation unit is included, and wherein air is employed as the source of oxygen for the primary reactor such that the C2 starting hydrocarbons are reacted essentially to extinction, and thus, a C2 recycle subsystem is eliminated.

As noted in the Background discussion of the present specification, oxychlorination is conventionally referenced as the oxidative addition of two chlorine atoms to ethylene from HCl or other reduced chlorine source. Catalysts capable of performing this chemistry have been classified as modified Deacon catalysts [Olah, G. A., Molnar, A., Hydrocarbon Chemistry, John Wiley & Sons (New York, 1995), pg 226]. Deacon chemistry refers to the Deacon reaction, the oxidation of HCl to yield elemental chlorine and water.

In contrast to oxychlorination, the preferred process described herein preferably utilizes oxydehydro-chlorination in converting ethane-containing and ethylene-containing streams to VCM at high selectivity. Oxydehydro-chlorination is the conversion of a hydrocarbon, using oxygen and a chlorine source, to a chlorinated hydrocarbon wherein the carbons either maintain their initial valence or have their valency reduced (that is, sp³ carbons remain sp³ or are converted to sp³, and sp² carbons remain sp² or are converted to sp). This differs from the conventional definition of oxychlorination whereby ethylene is converted to 1,2-dichloroethane, using oxygen and a chlorine source, with a net increase in carbon valency (that is, sp² carbons are converted to sp³ carbons). Given the ability of the catalyst to convert ethylene to vinyl chloride, it is advantageous to recycle ethylene produced in the oxydehydro-chlorination reaction process back to the reactor. The byproducts produced in the oxydehydro-chlorination reactor include ethyl chloride, 1,2-dichloroethane, cis-1,2-dichloroethylene and trans-1,2-dichloroethylene. The oxydehydro-chlorination catalyst is also an active catalyst for the elimination of HCl from saturated chlorohydrocarbons. Recycle of ethyl chloride and 1,2-dichloroethane is, in some cases, advantageously employed in the production of vinyl chloride. The remaining significant chlorinated organic side-products are the dichloroethylenes. These materials are, in one embodiment, hydrogenated to yield 1,2-dichloroethane. 1,2-dichloroethane is a large volume chemical and is either sold or recycled. In an alternative embodiment, EDC is hydrogenated completely to yield ethane and HCl. Intermediate severity hydrogenations yield mixtures of 1,2-dichloroethane, ethane, ethyl chloride, and HCl; such mixtures are also appropriate for recycle to the oxydehydro-chlorination reactor.

In one aspect, this invention provides a method of manufacturing vinyl chloride, comprising: (a) combining reactants including ethane, ethylene, or mixtures thereof with an oxygen source and a chlorine source in a reactor containing a suitable catalyst under conditions sufficient to convert substantially all of the C2 hydrocarbon fed and to produce a product stream comprising vinyl chloride and hydrogen chloride; and (b) recycling unreacted hydrogen chloride back for use in the combining step. For the purposes of this invention, the phrase “under conditions sufficient to convert substantially all of the C2 hydrocarbon fed” shall mean that greater than about 95 mole percent, preferably, greater than about 97 mole percent, of the C2 hydrocarbon fed (ethane, ethylene, or mixtures thereof) is converted to products. Accordingly, the unconverted C2 hydrocarbon shall comprise no greater than about 5 mole percent, preferably, no greater than about 3 mole percent, of a non-condensable lights stream obtained from the effluent of the reactor in step (a). Preferably, under these conditions residual unconverted C2 hydrocarbon, if any, need not be recovered; and therefore, a C2 recycle subsystem back to the reactor of step (a) is preferably eliminated from the process. The process of this invention can be run using air as an oxygen source, as explained in detail hereinafter. Further details of this invention are set forth in the description to follow and the Figures associated therewith, particularly FIGS. 5, 5a, and 5b.

In another embodiment of the first aspect of this invention, the product stream contains cis/trans-1,2-dechloroethylenes, which are hydrogenated with hydrogen in a hydrogenation reactor to form 1,2-dichloroethane (EDC), which itself is recycled, at least in part, to the oxydehydro-chlorination reactor of step (a).

In another aspect, this invention provides a method of manufacturing vinyl chloride, comprising: (a) combining reactants including ethane, optionally ethylene, an oxygen source, and a chlorine source in a reactor containing a suitable catalyst under conditions sufficient to produce vinyl chloride and hydrogen chloride; (b) catalytically reacting essentially all of said hydrogen chloride in a second reactor to provide a second reactor effluent essentially devoid of hydrogen chloride; and (c) recycling said second reactor effluent to catalytically react together with said ethane, said optional ethylene, said oxygen source, and said chlorine source in said combining step. For the purposes of this invention, the phrase “essentially devoid of hydrogen chloride” shall mean that essentially all of the hydrogen chloride is consumed in step (b), such that unconverted hydrogen chloride, if any at all, falls below a recoverable concentration. Additional features and advantages of the second aspect of the invention are set forth hereinafter and illustrated in the figures, particularly, FIGS. 6, 6a-6c, 7, and 7a-7c.

In a preferred embodiment of the second aspect of this invention, the hydrogen chloride recovered from the oxydehydro-chlorination effluent stream of step (a) is employed in a conventional oxychlorination reactor, for example those typical of the prior art, to convert ethylene in the presence of oxygen and the hydrogen chloride to ethylene dichloride, which itself is recycled to the primary oxydehydro-chlorination reactor of step (a).

In another preferred embodiment of the second aspect of this invention, the hydrogen chloride recovered from the oxydehydro-chlorination effluent stream of step (a) is employed in a liquid phase aqueous oxychlorination process wherein ethylene in the presence of oxygen and the hydrogen chloride is converted to ethylene dichloride, which itself is recycled to the primary oxydehydro-chlorination reactor (a).

In yet another preferred embodiment of the second aspect of this invention, the oxygen source is air, and the C2 hydrocarbon fed in step (a) (that is, ethane and optionally ethylene) is reacted essentially to extinction, that is, greater than 95 mole percent, and preferably, greater than about 97 mole percent, of the C2 hydrocarbon fed is converted to products. More preferably, in this embodiment there is essentially no recoverable unconverted C2 starting hydrocarbon for recycle back to the primary oxydehydro-chlorination reactor. Accordingly, more preferably, there is no need for a C2 hydrocarbon recovery subsystem.

In yet another preferred embodiment of the second aspect of this invention, the product stream contains cis/trans-1,2-dechloroethylenes, which are hydrogenated with hydrogen in a hydrogenation reactor to 1,2-dichloroethane (EDC), which itself is recycled, at least in part, to the oxydehydro-chlorination reactor.

Turning now to consideration of FIG. 1, for ethane-to-vinyl conversion as best understood from earlier publications, Ethane to VCM Process 100 shows characterization of a contemplated ethane-to-vinyl chloride process employing a catalyst capable of converting ethane to VCM; in this regard, the process does not provide for input of significant quantities of ethylene from either recycle streams or feed-streams to the ethane-VCM reactor (Ethane Reactor 102). It should also be noted that, since an ethane-to-vinyl manufacturing system of appropriate normal manufacturing scale has not, to the best knowledge of the inventors, been yet constructed, proposed process approaches are the only sources for embodiments which have been previously conceptualized. In this regard, Process 100 is a unified and simplified approximation to processes collectively reviewed in several publications respective to investigations and developments at EVC Corporation: Vinyl Chloride/Ethylene Dichloride 94/95-5 (August, 1996; Chemical Systems, Inc.; Tarrytown, New York); EP 667,845; U.S. Pat. No. 5,663,465; U.S. Pat. No. 5,728,905; and U.S. Pat. No. 5,763,710.

In consideration of the details shown in FIG. 1, Ethane Reactor 102 outputs a fluid stream to Quench Column 106 where HCl is quenched from the reactor output effluent. Quench Column 106 forwards a raw strong HCl aqueous stream to Phase Separation Subsystem 108. Phase Separation Subsystem 108 outputs a fluid stream to Anhydrous HCl Recovery Subsystem 110 where aqueous hydrogen chloride (hydrochloric acid), anhydrous HCl, and water are separated from the raw strong HCl aqueous stream.

Anhydrous HCl Recovery Subsystem 110 outputs Stream 130 to recycle anhydrous hydrogen chloride to Ethane Reactor 102, and Anhydrous HCl Recovery Subsystem 110 also outputs water (for subsequent use or to waste recovery). Anhydrous HCl Recovery Subsystem 110 returns a relatively dilute aqueous stream of HCl (hydrochloric acid) via Stream 128 to Quench Column 106. Quench Column 106 also outputs a fluid stream to Lights Column 114 where a lights stream containing ethylene is further removed from the reactor effluent product stream.

Lights Column 114 outputs the lights stream to Direct Chlorination Reactor 112 where chlorine (Stream 126) is added to directly chlorinate ethylene in the lights stream into EDC (1,2-dichloroethane). EDC is recovered in EDC Recovery Column 116 for recycle to Ethane Reactor 102, and a certain amount of the remaining lights gas is recycled to Ethane Reactor 102 as Stream 134 with CO (carbon monoxide) composition instrumentation providing a measurement (not shown) for use in a control system's (not shown) determination of an appropriate portion of the remaining lights gas for processing via Vent Oxidation Unit 118 to generate a vent stream for removal of CO, CO₂, and other impurities from the system.

Effluent from Lights Column 114 which does not proceed to Direct Chlorination Reactor 112 forwards (a) first, to Drying Subsystem 120 for removal of water; (b) further, to VCM Purification Column 122 for separation of VCM (vinyl chloride monomer) product; and then (c) further, to Heavies Column 124 for removal of heavies and generation of Stream 132. Stream 132 is a blended fluid of cis-1,2-dichloroethylene and trans-1,2-dichloroethylene, 1,2-dichloroethane, ethyl chloride, and other chlorinated organics. In an alternative contemplated embodiment based upon consideration of the literature, Drying Subsystem 120 removes water prior to Lights Column 114, with the VCM-carrying effluent from Lights Column 114 being forwarded (a) first, to VCM Purification Column 122 for separation of VCM (vinyl chloride monomer) product and then (b) further, to Heavies Column 124 for removal of heavies and generation of Stream 132.

Finally, Stream 132 forwards to RCl (chlorinated organics) Hydrogenation Reactor 104 where addition of hydrogen effects a recycle stream for forwarding to Ethane Reactor 102.

Turning now to consideration of FIGS. 2, 3, and 4, there are provided various embodiments of converting ethane, ethylene, or mixtures thereof with an oxygen source and a chlorine source to vinyl chloride monomer (VCM) in a one-step reactor. In contrast, to the prior art process of FIG. 1, FIGS. 2, 3, and 4 illustrate a one-step process that eliminates the need for an ethane to ethylene cracker by typically using a preferred rare earth catalyst, as described hereinafter. Moreover, FIGS. 2, 3, and 4 typically operate in a “fuel-rich” regime where unconverted hydrocarbon feed (ethane and/or ethylene) is recovered and recycled. Morever, HCl present in the product stream is recovered and recycled in the process of FIG. 2, or alternatively, reacted to extinction or near-extinction in the process of FIGS. 3 and 4. The present invention described herein provides modifications to the generalized one-step process illustrated in FIGS. 2, 3, and 4. Accordingly, the process embodiments of FIGS. 2, 3, and 4 are described in detail hereinafter.

Ethane to VCM Oxydehydro-chlorination Process 200 shows an ethane/ethylene-to-vinyl chloride process employing a catalyst capable of converting ethane and ethylene to VCM via oxydehydro-chlorination; in this regard, the process provides for input of significant quantities of both ethane and ethylene from either recycle streams or feed-streams to the reactor (Ethane/Ethylene To VCM Oxydehydro-chlorination Reactor 202). Ethane/Ethylene To VCM Oxydehydro-chlorination Reactor 202 receives input from (a) feed streams Ethane Feed Stream 222, HCl Feed Stream 224, Oxygen Feed Stream 226, and Chlorine Feed Stream 228 and (b) recycle streams Ethyl Chloride Stream 230, Hydrogen chloride (HCl) Stream 266, and Lights Recycle Stream 248 as well a portion of EDC Stream 262 when EDC is advantageously used for recycle according to the market and operational conditions at a particular moment of manufacture.

As reflected in The Dow Chemical Company, Patent Case No. 44649 to Mark E. Jones, Michael M. Olken, and Daniel A. Hickman, entitled “A PROCESS FOR THE CONVERSION OF ETHYLENE TO VINYL CHLORIDE, AND NOVEL CATALYST COMPOSITIONS USEFUL FOR SUCH PROCESS”, filed on Oct. 3, 2002, as International Patent Application serial no. PCT/00/27272 (published on May 31, 2001 as WO 01/38273), the catalyst used in Ethane/Ethylene To VCM Oxydehydro-chlorination Reactor 202 comprises at least one rare earth material. The rare earths are a group of 17 elements consisting of scandium (atomic number 21), yttrium (atomic number 39) and the lanthanides (atomic numbers 57-71) [James B. Hedrick, U.S. Geological Survey—Minerals Information—1997, “Rare-Earth Metals”]. The catalyst can be provided as either a porous, bulk material or it can be supported on a suitable support. Preferred rare earth materials are those based on lanthanum, cerium, neodymium, praseodymium, dysprosium, samarium, yttrium, gadolinium, erbium, ytterbium, holmium, terbium, europium, thulium, and lutetium. Most preferred rare earth materials for use in the aforementioned VCM process are based on those rare earth elements that are typically considered as being single valency materials. Catalytic performance of multi-valency materials appears to be less desirable than those that are single valency. For example, cerium is known to be an oxidation-reduction catalyst having the ability to access both the 3⁺ and 4⁺ stable oxidation states. This is one reason why, if the rare earth material is based on cerium, the catalyst further comprises at least one more rare earth element other than cerium. Preferably, if one of the rare earths employed in the catalyst is cerium, the cerium is provided in a molar ratio that is less than the total amount of other rare earths present in the catalyst. More preferably, however, substantially no cerium is present in the catalyst. By “substantially no cerium” it is meant that any cerium is in an amount less than 33 atom percent of the rare earth components, preferably less than 20 atom percent, and most preferably less than 10 atom percent.

The rare earth material for the catalyst is more preferably based upon lanthanum, neodymium, praseodymium or mixtures of these. Most preferably, at least one of the rare earths used in the catalyst is lanthanum. Furthermore, the catalyst is substantially free of iron and copper, especially as regards the ethylene feed. In general, the presence of materials that are capable of oxidation-reduction (redox) is undesirable for the catalyst. It is preferable for the catalyst to also be substantially free of other transition metals that have more than one stable oxidation state. For example, manganese is another transition metal that is preferably excluded from the catalyst. By “substantially free” it is meant that the atom ratio of rare earth element to redox metal in the catalyst is greater than 1, preferably greater than 10, more preferably greater than 15, and most preferably greater than 50.

As stated above, the catalyst may also be deposited on an inert support. Preferred inert supports include alumina, silica gel, silica-alumina, silica-magnesia, bauxite, magnesia, silicon carbide, titanium oxide, zirconium oxide, zirconium silicate, and combinations thereof. However, in a most preferred embodiment, the support is not a zeolite. When an inert support is utilized, the rare earth material component of the catalyst typically comprises from 3 weight percent (wt percent) to 85 wt percent of the total weight of the catalyst and support. The catalyst may be supported on the support using methods already known in the art.

It may also be advantageous to include other elements within the catalyst in both of the porous, bulk material and supported forms. For example, preferable elemental additives include alkaline earths, boron, phosphorous, sulfur, silicon, germanium, titanium, zirconium, hafnium, aluminum, and combinations thereof. These elements can be present to alter the catalytic performance of the composition or to improve the mechanical properties (for example attrition-resistance) of the material.

Prior to combining the ethane-containing, ethylene-containing, or ethane/ethylene-containing feed, oxygen source, and chlorine source in the reactor for the VCM process embodiment of this invention, it is preferable for the catalyst composition to comprise a salt of at least one rare earth element, with the proviso that the catalyst is substantially free of iron and copper and with the further proviso that when cerium is employed the catalyst further comprises at least one more rare earth element other than cerium. The salt of at least one rare earth element is preferably selected from rare earth oxychlorides, rare earth chlorides, rare earth oxides, and combinations thereof, with the proviso that the catalyst is substantially free of iron and copper and with the further proviso that when cerium is used the catalyst further comprises at least one more rare earth element other than cerium. More preferably, the salt comprises a rare earth oxychloride of the formula MOCl, wherein M is at least one rare earth element chosen from lanthanum, cerium, neodymium, praseodymium, dysprosium, samarium, yttrium, gadolinium, erbium, ytterbium, holmium, terbium, europium, thulium, lutetium, or mixtures thereof, with the proviso that, when cerium is present, at least one more rare earth element other than cerium is also present. Most preferably, the salt is a porous, bulk lanthanum oxychloride (LaOCl) material. This material beneficially does not undergo gross changes (for example, fracturing) when chlorinated in situ in this process, and provides the further beneficial property of water solubility in the context of this process after a period of use (LaOCl is initially water-insoluble), so that should spent catalyst need to be removed from a fluidized bed, fixed bed reactor or other process equipment or vessels, this can be done without hydroblasting or conventional labor-intensive mechanical techniques, by simply flushing the spent catalyst from the reactor in question with water.

Typically, when the salt is a rare earth oxychloride (MOCl), it has a BET surface area of at least 12 m²/g, preferably at least 15 m²/g, more preferably at least 20 m²/g, and most preferably at least 30 m²/g. Generally, the BET surface area is less than 200 m²/g. For these above measurements, the nitrogen adsorption isotherm was measured at 77K and the surface area was calculated from the isotherm data utilizing the BET method (Brunauer, S., Emmett, P. H., and Teller, E., Journal of the American Chemical Society, 60, 309 (1938)). In addition, it is noted that the MOCl phases possess characteristic powder X-Ray Diffraction (XRD) patterns that are distinct from the MCl₃ phases.

It is also possible to have mixtures of the rare earths (“M”) within the MOCl composition. For example, M can be a mixture of at least two rare earths selected from lanthanum, cerium, neodymium, praseodymium, dysprosium, samarium, yttrium, gadolinium, erbium, ytterbium, holmium, terbium, europium, thulium and lutetium. Similarly, it is also possible to have mixtures of different MOCl compositions wherein M is different as between each composition of the MOCl's in the mixture.

Once the ethane-containing, ethylene-containing or ethane/ethylene containing feed, oxygen source, and chlorine source are combined in the reactor, a catalyst is formed in situ from the salt of at least one rare earth element. In this regard, it is believed that the in situ formed catalyst comprises a chloride of the rare earth component. An example of such a chloride is MCl₃, wherein M is a rare earth component selected from lanthanum, cerium, neodymium, praseodymium, dysprosium, samarium, yttrium, gadolinium, erbium, ytterbium, holmium, terbium, europium, thulium, lutetium and mixtures thereof, with the proviso that when cerium is present the catalyst further comprises at least one more rare earth element other than cerium. Typically, when the salt is a rare earth chloride (MCl₃), it has a BET surface area of at least 5 m²/g, preferably at least 10 m²/g, more preferably at least 15 m²/g, more preferably at least 20 m²/g, and most preferably at least 30 m²/g.

In light of the disclosure herein, those of skill in the art will undoubtedly recognize alternative methods for preparing useful catalyst compositions. One method for forming the composition comprising the rare earth oxychloride (MOCl) comprises the following steps: (a) preparing a solution of a chloride salt of the rare earth element or elements in a solvent comprising either water, an alcohol, or mixtures thereof; (b) adding a nitrogen-containing base to cause the formation of a precipitate; and (c) collecting, drying and calcining the precipitate in order to form the MOCl material. Typically, the nitrogen-containing base is selected from ammonium hydroxide, alkyl amine, aryl amine, arylalkyl amine, alkyl ammonium hydroxide, aryl ammonium hydroxide, arylalkyl ammonium hydroxide, and mixtures thereof. The nitrogen-containing base may also be provided as a mixture of a nitrogen-containing base with other bases that do not contain nitrogen. In one embodiment, the nitrogen-containing base is tetra-alkyl ammonium hydroxide. The solvent in Step (a) may be water. Drying of the catalytically-useful composition can be done in any manner, including by spray drying, drying in a purged oven and other known methods. For a fluidized bed mode of operation, a spray-dried catalyst can be employed.

Another method for forming the catalyst composition comprising the rare earth chloride (MCl₃) comprises the following steps: (a) preparing a solution of a chloride salt of the rare earth element or elements in a solvent comprising either water, an alcohol, or mixtures thereof; (b) adding a nitrogen-containing base to cause the formation of a precipitate; (c) collecting, drying and calcining the precipitate; and (d) contacting the calcined precipitate with a chlorine source. For example, one application of this method (using La to illustrate) would be to precipitate LaCl₃ solution with a nitrogen containing base, dry it, add it to the reactor, heat it to 400° C. in the reactor to perform the calcination, and then contact the calcined precipitate with a chlorine source to form the catalyst composition in situ in the reactor.

Ethane/Ethylene To VCM Oxydehydro-chlorination Reactor 202 catalytically reacts together ethane, ethylene, hydrogen chloride, oxygen, and chlorine along with at least one recycle stream to yield Reactor Effluent Stream 232; and it is of special note that the molar ratio of ethane to ethylene derived from all feeds to Ethane/Ethylene To VCM Oxydehydro-chlorination Reactor 202 is between 0.02 and 50 (note that the particular operational ratio at any moment is determined by issues in operational process status) without long-term detriment to catalyst functionality. Depending on market and operational conditions at a particular moment of manufacture, ethylene is added to Reactor 202 via Ethylene Stream 289. In this regard, a more preferred molar ratio of ethane to ethylene derived from all feeds to Ethane/Ethylene To VCM Oxydehydro-chlorination Reactor 202 is between 0.1 and 10. When market and operational conditions (at a particular moment of manufacture) permit, one mode is for Ethylene Stream 289 to have a flow of zero and for the molar ratio of ethane to ethylene derived from all feeds to Ethane/Ethylene To VCM Oxydehydro-chlorination Reactor 202 to be between 1 and 6, with variance therein dependent upon local process conditions and catalyst life-cycle considerations. Even as the Reactor 202 effluent stream (Stream 232) is generated by catalytically reacting together ethane, ethylene, oxygen, and at least one chlorine source of hydrogen chloride, chlorine, or a saturated chlorohydrocarbon, it is to be noted that catalyst selectivity in the conversion of these streams to VCM benefits by, first, conditioning lanthanide-based catalysts with elemental chlorine. Catalyst selectivity in the conversion of these streams to VCM using lanthanide-based catalysts also benefits when elemental chlorine (Steam 228) is included as a portion of the chlorine source to Reactor 202. It should also be noted that any other catalyst systems, which exhibit the capacity to convert both ethane and ethylene to VCM, are advantageously, in alternative embodiments, also used with the VCM process and apparatus herein disclosed.

Chlorine sources (selected from hydrogen chloride, chlorine, and a saturated chlorohydrocarbon) HCl Feed Stream 224, Chlorine Feed Stream 228, any portion of EDC Stream 262 chosen for recycle, and any other recycled or raw material feed streams containing, without limitation, at least one of a chlorinated methane or a chlorinated ethane (for example, without limitation, carbon tetrachloride, 1,2-dichloroethane, ethyl chloride, 1,1-dichloroethane, and 1,1,2-trichloroethane) collectively provide chlorine to the oxydehydro-chlorination reaction; these streams are individually variable from moment to moment in real-time operation for providing the stoichiometric chlorine needed for VCM conversion. With respect to EDC from EDC Stream 262, market conditions affecting the opportunity for direct sale determine the appropriate amount for either recycle to Reactor 202 or direct sale. A further option for use of a portion of EDC Stream 262, dependent upon the particular facility, is for feedstock to a VCM conversion furnace. In this regard, operation of Process 200 is alternatively conducted so that (a) 1,2-dichloroethane generated in Reactor 202 is purified for sale, (b) 1,2-dichloroethane generated in Reactor 202 is purified for recycle to Reactor 202, and/or (c) 1,2-dichloroethane generated Reactor 202 is purified for cracking in a vinyl furnace. It is also to be noted the EDC is also, at occasional times, advantageously purchased for use as a chlorine source.

Ethane/Ethylene To VCM Oxydehydro-chlorination Reactor 202 outputs Reactor Effluent Stream 232 to feed Cooling Condenser 204. Cooling Condenser 204 treats Reactor Effluent Stream 232 to provide (a) a raw product (vapor) stream having a first portion of hydrogen chloride and (b) a raw cooled (aqueous) hydrogen chloride stream having the remainder of the hydrogen chloride which exited Reactor 202; the raw product (vapor) stream is Stream 240.

Stream 234 is conveyed to Phase Separation Subsystem 206 for removal of residual organic compounds from the raw cooled HCl. Phase Separation Subsystem 206 is, in alternative embodiments, a decanter, a stripper, or a combination of a decanter and stripper. From Phase Separation Subsystem 206 the removed organic materials (essentially in liquid phase) are conveyed to Lights Column 210 via Stream 242, and the separated raw cooled (essentially aqueous liquid) HCl is conveyed as Stream 236 to Anhydrous HCl Recovery Subsystem 208. Anhydrous HCl Recovery Subsystem 208 receives (aqueous) Stream 274 from Vent Oxidation Unit 214 (a thermal oxidation or other oxidation unit useful for vent stream purification to acceptable environmental compositions), and (aqueous) Stream 236 and generates output stream 266 as anhydrous HCl recycle to Ethane/Ethylene To VCM Oxydehydro-chlorination Reactor 202. Stream 268 outputs water from Anhydrous HCl Recovery Subsystem 208 for subsequent use or to waste recovery. In summary, Anhydrous HCl Recovery Subsystem 208 provides functionality to recover an anhydrous hydrogen chloride stream from the raw cooled hydrogen chloride stream and other aqueous HCl streams of Process 200. Anhydrous HCl Recovery Subsystem 208 also recycles the anhydrous hydrogen chloride (vapor) stream via HCl Stream 266 to the reactor 202. As should be apparent to those of skill, there are other methodologies for separating anhydrous HCl from mixtures of water and HCl.

Cooling Condenser 204 also outputs Stream 240 (vapor) to Lights Column 210 where a lights stream (vapor Stream 244) containing ethylene is further removed from the reactor effluent product stream.

After separation of HCl and lights stream (Stream 244) from the reactor effluent, Lights Column 210 forwards Stream 252 for separation of a water product stream (Stream 256), a vinyl chloride monomer product stream (Stream 254), an ethyl chloride stream (Stream 230), a cis-1,2-dichloroethylene and trans-1,2-dichloroethylene blended stream (Stream 260), a 1,2-dichloroethane stream (Stream 262), and a heavies stream (Stream 264). The manner of effecting these final separations is apparent to those of skill, and a substantial number of classically-utilized process units can be deployed in various configurations to achieve these separations. Drying Subsystem 216, VCM Purification Column 218, and Heavies Column 220 conveniently depict, therefore, the general separation systems (and, as such, should have the term “column” interpreted as a “virtual column” representing at least one physical column, although, in one contemplated embodiment, each column could be only a single physical column) for separation of Water Stream 256, VCM Product Stream 254, Ethyl Chloride Stream 230, Cis/trans-1,2-dichloroethylene Stream 260, and EDC Stream 262, with Heavies Stream 264 as organic material for destruction in a waste organic burner or use in an appropriate product where the general properties of Heavies Stream 264 are acceptable. In an alternative contemplated embodiment, Drying Subsystem 216 removes water prior to Lights Column 210, with the effluent from Lights Column 210 being forwarded to VCM Purification Column 218. Note again that, with respect to EDC from EDC Stream 262, market conditions affecting the opportunity for direct sale function to determine the appropriate amount for either recycle to Reactor 202 or direct sale. In this regard, operation of VCM Purification Column 218, and Heavies Column 220 is alternatively conducted so that (a) 1,2-dichloroethane is purified for sale, (b) 1,2-dichloroethane is purified for recycle to Reactor 202, and/or (c) 1,2-dichloroethane is purified for cracking in a vinyl furnace.

Returning now to Stream 244 as it exists from Lights Column 210, Stream 244 is forwarded to Ethylene Oxychlorination Reactor 282 where oxygen is added and an oxychlorination reaction effected with a traditional oxychlorination catalyst to consume the bulk of HCl and generate EDC. The output from Ethylene Oxychlorination Reactor 282 is forwarded as Stream 284 to Residual HCl Treatment Unit 286 which scrubs any residual HCl from Stream 284 and outputs essentially an essentially aqueous stream with some HCl as Stream 288 to waste treatment. Residual HCl Treatment Unit 286 also outputs stream 290 to EDC Column 292 where Crude EDC Stream 294 is separated and forwarded to Drying Subsystem 216.

Output from EDC Column 292 (Stream 273) is divided into a first stream portion forwarded directly in Stream 248 to Ethane/Ethylene To VCM Oxydehydro-chlorination Reactor 202 and into a second stream which forwards to C2 Absorption and Stripping Columns 212. C2 Absorption and Stripping Columns 212 absorb and strip C2 materials (ethane and ethylene) from the forwarded second stream portion of Stream 244 and insure the recycle of the C2 materials to Reactor 202 via C2 Recycle Stream 246 which, in combination the first stream portion from Stream 244, forms Stream 248. C2 Absorption and Stripping Columns 212 also outputs a purge stream to Vent Oxidation Unit 214 which outputs Vent Stream 250 to the atmosphere and also (aqueous) Stream 274 to Anhydrous HCl Recovery Subsystem 208. CO (carbon monoxide) composition instrumentation provides a measurement (not shown) for use in a control system's (not shown) determination of an appropriate portion of the remaining lights gas for processing via C2 Absorption and Stripping Columns 212 and Vent Oxidation Unit 214 to generate Vent Stream 250 so that CO does not accumulate to unacceptable levels in the process.

Simulated relative stream flows and stream compositions for Ethane to VCM Oxydehydro-chlorination Process 200 are appreciated from a consideration of Table 1. Table 1 (mass unit/time unit) data uses laboratory-derived catalyst performance measurements for lanthanum oxychloride at 400 degrees Celsius and essentially ambient pressure; further details on the preferred catalyst are appreciated from a study of “A PROCESS FOR THE CONVERSION OF ETHYLENE TO VINYL CHLORIDE, AND NOVEL CATALYST COMPOSITIONS USEFUL FOR SUCH PROCESS,” referenced hereinabove. Table 1 shows some flows as a zero in the context of the simulation generating the data, but such a numeric value is not intended to mean a total absence of flow or absence of need for a stream. Table 1 does not show Ethylene Feed Stream 289; in this regard, and reprising an earlier point, when market and operational conditions at a particular moment of manufacture permit, the most preferred mode is for Ethylene Stream 289 to have a flow of zero. However, under certain conditions, Ethylene Stream 289 does contribute an economically beneficial flow.

TABLE 1
ETHANE/ETHYLENE TO VINYL CHLORIDE MASS BALANCE FOR PROCESS 200

| Stream | C₂H₆ | C₂H₄ | O₂ | HCl | Cl₂ | Ar | CO | CO₂ | EDC | EtCl | VCM | DCE | H₂O | total |
|---|---|---|---|---|---|---|---|---|---|---|---|---|---|---|
| 222 | 573 | 0 | 0 | 0 | 0 | 0 | 0 | 0 | 0 | 0 | 0 | 0 | 0 | 573 |
| 224 | 0 | 0 | 0 | 0 | 0 | 0 | 0 | 0 | 0 | 0 | 0 | 0 | 0 | 0 |
| 226 | 0 | 0 | 539 | 0 | 0 | 5 | 0 | 0 | 0 | 0 | 0 | 0 | 0 | 545 |
| 228 | 0 | 0 | 0 | 0 | 639 | 0 | 0 | 0 | 0 | 0 | 0 | 0 | 0 | 639 |
| 230 | 0 | 0 | 0 | 0 | 0 | 0 | 0 | 0 | 0 | 45 | 0 | 0 | 0 | 45 |
| 232 | 895 | 456 | 32 | 732 | 0 | 42 | 709 | 129 | 163 | 45 | 1000 | 95 | 499 | 4798 |
| 234 | 40 | 12 | 0 | 456 | 0 | 0 | 1 | 3 | 157 | 31 | 428 | 87 | 494 | 1709 |
| 236 | 0 | 0 | 0 | 456 | 0 | 0 | 0 | 0 | 0 | 0 | 0 | 0 | 494 | 950 |
| 240 | 855 | 444 | 32 | 276 | 0 | 42 | 708 | 126 | 6 | 14 | 572 | 8 | 5 | 3089 |
| 242 | 40 | 12 | 0 | 0 | 0 | 0 | 1 | 3 | 157 | 31 | 428 | 87 | 0 | 760 |
| 244 | 895 | 456 | 32 | 276 | 0 | 42 | 709 | 129 | 0 | 0 | 0 | 0 | 0 | 2541 |
| 246 | 109 | 43 | 0 | 0 | 0 | 0 | 0 | 0 | 0 | 0 | 0 | 0 | 0 | 152 |
| 248 | 889 | 349 | 0 | 241 | 0 | 37 | 618 | 113 | 0 | 0 | 0 | 0 | 0 | 2247 |
| 250 | 0 | 0 | 0 | 0 | 0 | 5 | 0 | 183 | 0 | 0 | 0 | 0 | 13 | 202 |
| 252 | 0 | 0 | 0 | 0 | 0 | 0 | 0 | 0 | 163 | 45 | 1000 | 95 | 0 | 1303 |
| 254 | 0 | 0 | 0 | 0 | 0 | 0 | 0 | 0 | 0 | 0 | 1000 | 0 | 0 | 1000 |
| 256 | 0 | 0 | 0 | 0 | 0 | 0 | 0 | 0 | 0 | 0 | 0 | 0 | 5 | 5 |
| 260 | 0 | 0 | 0 | 0 | 0 | 0 | 0 | 0 | 0 | 0 | 0 | 95 | 0 | 95 |
| 262 | 0 | 0 | 0 | 0 | 0 | 0 | 0 | 0 | 534 | 0 | 0 | 0 | 0 | 534 |
| 264 | 0 | 0 | 0 | 0 | 0 | 0 | 0 | 0 | 0 | 0 | 0 | 0 | 0 | 0 |
| 266 | 0 | 0 | 0 | 491 | 0 | 0 | 0 | 0 | 0 | 0 | 0 | 0 | 0 | 491 |
| 268 | 0 | 0 | 0 | 0 | 0 | 0 | 0 | 0 | 0 | 0 | 0 | 0 | 494 | 494 |
| 278 | 0 | 0 | 0 | 35 | 0 | 0 | 0 | 0 | 0 | 0 | 0 | 0 | 75 | 111 |
| 284 | 895 | 351 | 0 | 3 | 0 | 42 | 709 | 129 | 371 | 0 | 0 | 0 | 68 | 2568 |
| 288 | 0 | 0 | 0 | 3 | 0 | 0 | 0 | 0 | 0 | 0 | 0 | 0 | 68 | 70 |
| 290 | 895 | 351 | 0 | 0 | 0 | 42 | 709 | 129 | 371 | 0 | 0 | 0 | 0 | 2498 |
| 294 | 0 | 0 | 0 | 0 | 0 | 0 | 0 | 0 | 371 | 0 | 0 | 0 | 0 | 371 |

Even as the embodiments of processes presented thus far have been made possible by developments in catalysis, catalytic development directions are suggested from further consideration of appropriate derived processes, which are viable in the context of catalyst systems that are capable of reacting ethane and ethylene while essentially fully reacting the HCl fed to Reactor 202. FIGS. 3 and 4 present other suggested ethane-to-vinyl chloride process embodiments in anticipation of catalytic developments that hopefully will enable essentially full HCl consumption in either two reactors in series or in a single reactor.

Turning now to FIG. 3, Ethane/Ethylene to VCM Oxydehydro-chlorination Dual Reactor System 300 modifies Ethane to VCM Oxydehydro-chlorination Process 200 to interpose Stage 2 Reactor 296 between Ethane/Ethylene To VCM Oxydehydro-chlorination Reactor 202 and Cooling Condenser 204. Second stage reactor 296 functions to react hydrogen chloride output to extinction by any means, such as, conventional oxychlorination or reaction of HCl with ethylene to make ethyl chloride. Output Lights Stream 244, from Product Split 210, is also divided into a first stream that is forwarded directly in Stream 248 to Ethane/Ethylene To VCM Oxydehydro-chlorination Reactor 202 and into a second stream that forwards to C2 Absorption and Stripping Columns 212. C2 Absorption and Stripping Columns 212 absorb and strip C2 materials from the forwarded portion of Stream 244 and ensure the recycle of the C2 materials to Reactor 202 via C2 Recycle Stream 246 and Stream 248. C2 Absorption and Stripping Columns 212 also outputs a purge stream to Vent Oxidation Unit 214, which outputs Vent Stream 250 to the atmosphere. Note that there is no need for Anhydrous HCl Recovery Subsystem 208 since essentially no HCl is present in Stage 2 Reactor 296 effluent.

FIG. 4 shows VCM-Furnace-Augmented Ethane/Ethylene to VCM Oxydehydro-chlorination Dual Reactor System 400 comprising Oxydehydro-chlorination Reactor 202 and Second Stage Reactor 296, with some HCl present in Stage 2 Reactor 296 effluent. Quench Column 204 treats Reactor Effluent Stream 232 to essentially completely remove residual HCl by quenching the reactor effluent stream to provide a raw product stream essentially devoid of hydrogen chloride. A raw cooled hydrogen chloride stream (Stream 234) is also output from Quench Column 204; Stream 234 is conveyed to Phase Separation Subsystem 206 for removal of organic compounds from the raw cooled HCl. The removed organic materials are conveyed to Lights Column 210 via Stream 242. Aqueous HCl is recycled from Phase Separation Subsystem 206 to Quench Column 204, and Neutralizer 298 treats the waste stream from Phase Separation Subsystem 206 with sodium hydroxide or another appropriate neutralization additive. System 400 also shows the embellishment of treating EDC Stream 262 in Vinyl Furnace System 293 and VCM Finishing System 295 to generate supplementary VCM product and also an anhydrous HCl stream to Reactor 202.

Turning now to a consideration of the embodiments of this invention, FIG. 5 illustrates the process invention where air is employed as the source of oxygen for the ethane, ethylene, or ethane/ethylene-to-vinyl chloride process. In FIG. 5, a C2 steam (ethane, ethylene, or both ethane and ethylene), HCl, air, and chlorine are introduced into Oxydehydro-chlorination Reactor 502 via lines 503, 504, 505, and 506, respectively. The HCl is optionally added via line 504, as needed or if available. HCl is also introduced as recycle from HCl Recovery unit 538 via line 508. Likewise, ethylene chloride (EtCl) can be recycled from the VCM purification unit 522 and introduced to the Reactor 502 via line 558. Ethylene dichloride (EDC) can also be recycled from the EDC purification unit 524 and introduced to Reactor 502 via line 560. The Reactor Effluent Stream 523 is cooled and condensed in unit 510 where the Effluent Stream 523 is treated to provide (a) a Raw Product (vapor) Stream having a first portion of hydrogen chloride that exits via line 511 and (b) a raw cooled (aqueous) hydrogen chloride stream having the remainder of the hydrogen chloride which exited Reactor 502 and which exits the Cooling Condenser 510 via line 512.

The raw cooled hydrogen chloride stream is treated in Phase Separation Subsystem 516 to remove residual organic compounds. The Phase Separation Subsystem may be an alternative embodiment discussed for FIG. 2. The residual organic compounds are conveyed to a Product Split 518 via line 517, with the separated raw cooled (essentially aqueous liquid) HCl being sent to the Anhydrous HCl Recovery Subsystem 538. Aqueous HCl is introduced to the HCl Recovery Subsystem 538 via line 539. Water exits the HCl Recovery Subsystem 538 via line 540. Recovered HCl (anhydrous) is recycled to the Reactor 502 via line 508. It should be appreciated that Anhydrous HCl Recovery Subsystem 538 provides functionality to recover an anhydrous hydrogen chloride stream from the raw cooled hydrogen chloride stream and other aqueous HCl streams from the Reactor 502. Anhydrous HCl Recovery Subsystem 538 also recycles the anhydrous hydrogen chloride (vapor) stream to Reactor 502. Typically, the HCl Recovery Subsystem employs a distillation process to recover the anhydrous HCl from the aqueous HCl streams. The anhydrous HCl may be recycled. As should be apparent to those of skill, there are other methodologies for separating anhydrous HCl from mixtures of water and HCl.

In Product Split 518, a lights stream is separated which exits via line 515. The lights stream contains ethylene and may include other components. The balance of the effluent, which contains VCM and may contain other components, is forwarded via line 516 to separation in series to the Drying Subsystem 520, VCM Purification 522, and EDC Purification 524. The manner of effecting these final separations is apparent to those of skill in the art and a substantial number of classic process units can be deployed in various configurations to achieve the separations. Drying Subsystem 520, VCM Purification 522, and EDC Purification 524 conveniently depict, therefore, the general separation systems for separation of Water Stream 556, VCM Product Stream 557, Ethyl Chloride Stream 558, Cis/trans-1,2-dichloroethylene Stream 559, and EDC Stream 560, with Heavies Stream 561 as organic material for destruction or use in an appropriate product where the general properties of Heavies Stream 561 are acceptable. In an alternative contemplated embodiment, Drying Subsystem 520 removes water prior to Product Split 518, with the effluent from Product Split 518 being forwarded to VCM Purification 522.

The lights stream from Product Split 518, which exited via line 515, is sent to HCl Absorption Subsystem 570. In the HCl Absorption Subsystem 570, an absorber may be used to removed trace amounts of HCl from the gaseous compounds and return the HCl to HCl Recovery Subsystem 538 such as through line 539. The HCl-stripped stream exits the HCl Absorption Subsystem via line 571 to Vent Oxidation Unit 580 where any remaining combustible compounds are converted to carbon dioxide (CO₂) which may be vented to the atmosphere. Alternatively, if the lights stream contains recoverable quantities of C2 hydrocarbons, such as ethylene or ethane, then appropriate means to recover those hydrocarbons can be interposed between the HCl adsorption unit and the Vent treatment. Collected hydrocarbons would then be returned to unit reactor 502. Preferably, however, the process produces essentially produces no recoverable quantity of C2 hydrocarbons.

In the configuration of FIG. 5, in a preferred embodiment, a relatively small amount of C2 hydrocarbon (ethane, ethylene, or mixture thereof) is input into Reactor 502 in comparison to the amount of air entering the system. This may be referred to as a “fuel lean” process. In such a configuration, the large amount of air produces a very high conversion of C2 hydrocarbon and with the chlorinated organic products being condensable. This results in a one-pass operation with a large amount of venting, with recycle of HCl. The rate of addition of the reactant C2 hydrocarbon, air, and chlorine source vary depending on conditions, and are readily determined by one of skill in the art. One of skill in the art would also appreciate that the process needs to be operated mindful of flammability issues that may arise and that may be different from the use of pure oxygen in a “fuel rich” process design. The benefits of running such an air-fed process includes the ability to run the process in areas where pure oxygen sources are difficult to obtain or where existing manufacturing equipment makes the use of an air-fed process desirable.

FIG. 5a illustrates another embodiment of this invention where air is employed as the source of oxygen for the ethane, ethylene, or ethane/ethylene-to-vinyl chloride process. The process scheme depicted in FIG. 5a is basically the same as that of FIG. 5, except that a RCl (chlorinated organic compounds) Hydrogenation Reactor 590 is included, where addition of hydrogen effects hydrogenation of the cis/trans-1,2-dichloroethylene from the EDC Purification to form ethylene dichloride, which can be recycled back to the Reactor 502 as a source of chlorine via line 591. EDC stream 560 may optionally be present to assist in balancing the concentration of cis- and trans-1,2-dichloroethylene fed to the Hydrogenation Reactor 590. It may be removed for sale, or may be recycled to Reactor 502. This process scheme has the advantage of producing a stream composed of a single compound, ethylene dichloride, rather than a mixed stream including the cis/trans-1,2-dichloroethylene, the former of which may be recycled back to the Reactor 502.

FIG. 5b illustrates another embodiment of this invention wherein the effluent from the Reactor 502 is fed directly to HCl Absorption Subsystem 570, which serves to separate a gaseous stream 571 that contains vinyl chloride and other components which is sent to the Cooling Condenser 510, and a second stream (the liquid phase) 572 that is sent to the Phase Separation Subsystem 516. Phase Separation Subsystem 516 serves to remove residual organic compounds. The Phase Separation Subsystem may be an alternative embodiment, as discussed for the previous figures. The residual organic compounds are conveyed to the Drying Subsystem 520, which proceeds as discussed above in the discussion of FIG. 5. The HCl-rich stream from Phase Separation Subsystem 516 is sent to HCl Recovery Subsystem 538 where HCl is isolated then recycled to Reactor 502 as stream 508.

FIG. 6 illustrates an alternative embodiment of this invention where HCl from the ethane-to-vinyl chloride or ethane/ethylene-to-vinyl chloride process is employed as a “wet” feed to a conventional oxychlorination reactor where a secondary source of oxygen and ethylene are fed to form ethylene dichloride, which is recycled back to the Oxydehydro-chlorination Reactor 602 in EDC line 643 with feed 660 of EDC being added, if desired. In FIG. 6, (i) ethane, (ii) HCl, (iii) oxygen, and (iv) chlorine are introduced into the Oxydehydro-chlorination Reactor 602 via lines 603, 605, and 606, respectively. Optionally, ethylene can be added to the feed to Reactor 602 via recycle Stream 672. HCl is optionally added via line 604. Likewise, ethylene chloride (EtCl) can be recycled from the VCM purification unit 622 and introduced to the Reactor 602 via line 658. The Reactor Effluent Stream 623 is cooled and condensed in unit 610 where the Effluent Stream 623 is treated to provide (a) a Raw Product (vapor) Stream having a first portion of hydrogen chloride that exits via line 611 and (b) a raw cooled (aqueous) hydrogen chloride stream having the remainder of the hydrogen chloride, which exited Reactor 602 and which exits the Cooling Condenser 610 via line 612.

The raw cooled hydrogen chloride stream is treated in Phase Separation Subsystem 616 to remove residual organic compounds. The Phase Separation Subsystem may be an alternative embodiment as discussed in the previous figures. The residual organic compounds are conveyed via line 617 to a Product Split 618, with the separated raw cooled (essentially aqueous liquid) HCl being sent to the Aqueous HCl Recovery Subsystem 638 via line 636. Aqueous HCl is introduced to the Aqueous HCl Recovery Subsystem 638 via line 639. In the Aqueous HCL Recovery Subsystem, water and HCl are vaporized as a constant boiling azeotropic mixture. The gaseous effluent is referred to as “wet” HCl, with the water basically functioning as a diluent for the HCl.

The recovered wet HCl (a gaseous mixture of water and HCl) is conveyed to Conventional Oxychlorination Reactor 642 via line 640, with oxygen and ethylene being fed to the Conventional Oxychlorination Reactor 642 via lines 646 and 647, respectively. It should be appreciated that a conventional oxychlorination reactor is one that operates a gas phase reaction using a catalyst such as a copper/alumina catalyst to effect conversion of the oxygen, ethylene, and source of chlorine to ethylene dichloride (EDC), as discussed in the Background of this invention. This process thus advantageously recycles HCl produced by the oxydehydro-chlorination reaction that occurs in Reactor 602, using it to make ethylene dichloride (EDC) which, along with EDC 660, can be recycled to the Reactor 602 via line 643, or can be sold. Before the EDC is sent to the Reactor 602, however, water is removed from the stream, which is denoted by line 644. Other waste products may be sent for Vent Treatment, as denoted by line 645.

In Product Split 618, a lights stream is separated which exits via line 615. The lights stream from Product Split 618, which contains ethylene and may include other components and which exited via line 615, is split and recycled to the Reactor 602 via Stream 672 and to HCl Absorption Subsystem 670. The balance of the effluent, which contains VCM and may contain other components such as ethyl chloride (EtCl), cis/trans-1,2-dichloroethylene, and ethylene dichloride, is forwarded via line 619 for separation in series to the Drying Subsystem 620, VCM Purification Column 622, and EDC Purification 624. The manner of effecting these final separations is apparent to those of skill in the art and a substantial number of classic process units can be deployed in various configurations to achieve the separations. Drying Subsystem 620, VCM Purification 622, and EDC Purification 624 conveniently depict, therefore, the general separation systems for separation of Water Stream 656, VCM Product Stream 657, Ethyl Chloride Stream 658, cis/trans-1,2-dichloroethylene Stream 659, and EDC Stream 660, with Heavies Stream 661 as organic material for destruction in a waste organic burner or use in an appropriate product where the general properties of Heavies Stream 661 are acceptable. In an alternative contemplated embodiment, Drying Subsystem 620 removes water prior to Product Split 618, with the effluent from Product Split 618 being forwarded to VCM Purification Column 622.

The lights stream from Product Split 618, which contains ethylene and may include other components; that exited via line 615 is split and recycled to the Reactor 602 and to HCl Absorption Subsystem 670. In the HCl Absorption Subsystem 670, an absorber may be used to remove trace amounts of HCl from the gaseous compounds and return the HCl to Aqueous HCl Vaporization Subsystem 638 such as through line 639. The HCl-stripped stream exits the HCl Absorption Subsystem 670 via line 671 to C2 Absorption and Stripper Subsystem. C2 Absorption and Stripping Columns 675 absorb and strip C2 materials (ethane and ethylene), then recycles the C2 materials to Reactor 602 via C2 Recycle Stream 676, which in combination with the first stream portion from line 615, forms Stream 672. C2 Absorption and Stripping Columns 675 also conveys a purge stream to Vent Treatment Unit 680 via line 681 where the vent is treated prior to release to the environment. This unit may provide means to convert any remaining combustible compounds to carbon dioxide (CO₂), which may be vented to the atmosphere. In such equipment, CO (carbon monoxide) composition instrumentation may provide a measurement (not shown) for use in a control system's (not shown) determination of an appropriate portion of the remaining lights gas for processing via C2 Absorption and Stripping Columns 675 and Vent Oxidation Unit 680 to generate Vent Stream 682, so that CO does not accumulate to unacceptable levels in the process.

The scheme of FIG. 6 has the advantage of separating and catalytically reacting HCl for conversion to EDC. The EDC can be recycled to the oxydehydro-chlorination reactor or may be used or otherwise sold. Notably, a hydrogen chloride recovery unit is eliminated from the scheme, which beneficially eliminates long-term corrosion problems.

FIG. 6a illustrates an alternative scheme to the embodiment of FIG. 6, wherein a hydrogenation step is included. Thus, the scheme is the same as in FIG. 6 except that all or a portion of the cis/trans-1,2-dichloroethylene recovered from EDC Column 624 is fed via line 625 to Hydrogenation 626 (a conventional hydrogenation unit) where hydrogen is fed via line 627 and wherein the cis/trans-1,2-dichloroethylene is converted to EDC. The EDC effluent exits via line 628. This EDC can be recycled to the reactor via, for instance, via line 628 to line 643 into Reactor 602. This configuration provides the advantage of alternatively forming EDC from the cis/trans-1,2-dichloroethylene, which may not be a desirable co-product of the reaction scheme.

FIG. 6b illustrates another alternative scheme to the embodiment of FIG. 6c, wherein a C2 absorption and stripper block and recycle is omitted. In this configuration, air is fed to Reactor via line 605. Hence, the process is operated in a fashion similar to the embodiment of the invention depicted in FIG. 5 such that “fuel lean” conditions are implemented with the ethane, ethylene, or both ethane and ethylene being reacted to extinction in a single-pass operation. The process thus is designed to treat a large volume of gas to the vent 680. Since ethane is essentially fully converted, there is no recycle of lights unlike the scheme in FIG. 6, but similar to the scheme of FIG. 5. It should be appreciated that effluent from HCl Absorption Subsystem is conveyed directly via line 671 to the Vent Treatment Unit 680, with vent stream exiting via line 682.

FIG. 6c illustrates an alternative scheme to the embodiment of FIG. 6b, wherein the C2 absorption and stripper block and recycle is again omitted but a hydrogenation unit 626 is included and the process is run using air instead of oxygen. This configuration has the advantage that HCl is recovered, and the cis/trans-1,2-dichloroethylene may be converted to EDC and recycled to the Reactor 602 or sold. This scheme employs conventional oxychlorination to assist in recycling the HCl by reacting it with ethylene and oxygen in the presence of an oxychlorination catalyst to form EDC.

FIG. 7 illustrates another alternative scheme to the embodiment of FIG. 6, wherein aqueous HCl (the HCl is formed in the primary reactor) serves as a reaction medium and reactant used to form ethyl chloride, ethylene dichloride, or mixtures of the two. Ethyl chloride and ethylene dichloride sold or can be fed to the primary Reactor 602 to provide a source of chlorine. FIG. 7 is the same scheme as FIG. 6, except that the raw cooled hydrogen chloride stream is treated in Phase Separation Subsystem 616 to remove residual organic compounds, with the residual organic compounds being conveyed via line 617 to a Product Split 618, and with the separated raw cooled (essentially aqueous liquid) HCl being sent with make up HCl to Reactor 690 (Kellogg process). The Reactor 690 may be operated in accordance with U.S. Pat. No. 3,214,482 and GB 1,063,284. Ethylene and oxygen are fed to the Reactor 690 via feed streams 647 and 646, respectively, which reactor may employ a catalyst composition comprising an aqueous solution of active metal halide and either a solubilizing agent or promoter. Active metals include, but are not limited to copper, as described in GB 1,063,284, and iron, as described in U.S. Pat. No. 3,214,482. The reaction of ethylene, oxygen, and HCl is typically run at a temperature of from about 10° C. to about 350° C., more typically from about 120° C. to about 180° C., under pressure sufficient to maintain the aqueous phase, using standard equipment and methodologies. The ethylene, oxygen, and HCl react to form ethyl chloride (EtCl), EDC, and water. The EDC and EtCl are separated from the water via phase separation. The EDC and EtCl are then fed to the Steam Stripper 694, with residual EDC and EtCl in the aqueous phase that is stripped from the aqueous phase exiting via line 695 to Vaporization Unit 692. The Vaporization Unit 692 is typically a heat exchange process that vaporizes the EDC and EtCl streams, with the resulting effluent being recycled to Reactor 602 via line 696. The remainder of the process flow depicted in FIG. 7 is described above in the description of FIG. 6. The process design of FIG. 7 has the advantage of including an alternative reactor for use in recovering HCl from the process in the form of EDC and/or EtCl, which can be recycled to the primary Reactor 602, or used, or sold directly.

FIG. 7a illustrates an alternative scheme to the embodiment of FIG. 7 wherein a hydrogenation step is included. FIG. 7a is identical to FIG. 7, except that a Hydrogenation Unit 626 is included. A discussion of Hydrogenation Unit 626 is described in the section above concerning FIG. 6a. An advantage to this process flow scheme is that the cis/trans-1,2-dichloroethylene is converted to EDC, which may be used or sold or which may be recycled to the Reactor 602 to thereby improve the overall yield of vinyl chloride monomer.

FIG. 7b illustrates an alternative scheme to the embodiment of FIG. 7 wherein air is employed as the source of oxygen for the primary reactor. This process flow scheme is the same as that of FIG. 7, with the exception that the C2 hydrocarbon absorption and stripper 675 and C2 hydrocarbon recycle streams 672/676 are absent. The scheme is operated with the ethane, ethylene, or both ethane and ethylene being reacted to extinction whereby there is no recycle of lights to Reactor 602 via line 672 as in FIG. 7a. In this case, air serves as the preferred oxidant.

FIG. 7c illustrates another alternative scheme for the embodiment of FIG. 7b. FIG. 7c is the same as FIG. 7b, except that a Hydrogenation Unit 626 is included, which converts a flow of cis/trans-1,2-dichloroethylene to EDC. The EDC can be sold or recycled as a chlorine source to Reactor 602. Table 2 presents further detail in components identified in the Figures. Certain unit features may be recognizably similar from Figure to Figure, although the numbers for the specific unit feature may be different on different figures.

TABLE 2
Component Detail

| Drawing | | |
|---|---|---|
| Element | Name | Description |
| 102 | Reactor | Fluid bed ethane and/or ethylene reactor. Vertically oriented |
| | | reactor system with gas feed at bottom and outlet at top. |
| | | Vertical cooling tubes in bed and internal cyclones (up to 3 in |
| | | series) located at the top. Typical diameters up to 20 feet. |
| | | Height of fluid bed 30 to 50 feet, with total height of 80 feet. |
| | | The reactor temperature of >400° C. requires that a high nickel |
| | | alloy be used for construction. |
| 104 | RCL Hydrogenation | Hydrogenation reactor for converting the unsaturated |
| | | compounds (most are chlorinated, such as cis-1,2 |
| | | dichloroethylene or trans-1,2 dichloroethylene) to their |
| | | saturated derivatives for recycle to the reactor 102. |
| 106 | Cool and Scrub | Product gas from the reactor is cooled and the condensate |
| | | separated from the vapor. The condensate has both a |
| | | concentrated HCl aqueous phase and an organic phase. |
| 108 | Phase Separate | Gravity separation of the aqueous and organic phases from |
| | | Cooler 106 is preferably achieved with a horizontal tank |
| | | provided with internal baffles to allow the heavy phase (most |
| | | likely the aqueous/acid phase, but the nature of the phases |
| | | depends on the exact composition of organics in the phases) to |
| | | be removed from one end of the vessel. The lighter phase |
| | | flows over the baffle into the second half of the vessel for |
| | | removal. The aqueous phase is then, in some embodiments, |
| | | stripped of organics. |
| 110 | HCl Recovery | The aqueous HCl stream from the separator is recovered as |
| | | anhydrous HCl for recycle to the reactor using traditionally |
| | | deployed approaches that are apparent to those of skill. |
| 112 | Direct Chlorination | Reactor for the chlorination of ethylene. This is typically |
| | | accomplished by injecting chlorine and ethylene into the |
| | | bottom of a vessel containing EDC. The reactants form EDC; |
| | | the net product removed as an overhead vapor. The heat of |
| | | reaction provides the driving force for the vaporization. |
| 114 | Product Split | Separation column with refrigerated condensers at the top to |
| | | allow separation of the lights for recycle from the chlorinated |
| | | organics. |
| 116 | EDC Recovery | Standard distillation columns for the purification of EDC. |
| 118 | Vent Treatment | Vent treatment is achieved with an incinerator for the |
| | | oxidation of organics (including chlorinated organics) to water |
| | | vapor, carbon dioxide, and hydrogen chloride. The vent gas is |
| | | scrubbed with water to recover HCl as a relatively dilute (10 |
| | | to 20% HCl stream) for other uses. This unit is typical of |
| | | those found throughout the chemical industry and should be |
| | | apparent to those of skill. |
| 120 | Drying | Prior to the final separation of the VCM from the other |
| | | products, water is removed in a drying column. The pressure |
| | | and temperature are adjusted such that the water is removed |
| | | from the bottom of the column and the dry product is removed |
| | | from the top. |
| 122 | VCM Columns | Final purification of the VCM product as practiced in industry. |
| 124 | Recycle Products | A distillation column to effect the separation of the cis and |
| | Column | trans 1,2 dichloroethylenes and EDC from the heavier (higher |
| | | molecular weight) components. The recovered components |
| | | are sent to the hydrogenation reactor prior to recycling to the |
| | | reactor. |
| 202 | Reactor | Ethylene/ethane oxydehydro-chlorination reactor. |
| | | A fluid bed version (preferred) of the reactor is a vertically |
| | | oriented reactor system with gas feed at bottom and with the |
| | | outlet at the top. Vertical cooling tubes are positioned in the |
| | | bed, and internal cyclones (up to 3 in series) are located at the |
| | | top. Typical diameter of the reactor is less than 20 feet. |
| | | Height of fluid bed is between 30 feet and 50 feet, with a total |
| | | height of 80 feet for the reactor. |
| | | The fixed bed version of the reactor is a vertical exchanger |
| | | type catalytic reactor with tubes from 1 to 1.5 inches. The |
| | | reactor temperature of >400° C. requires that a high nickel |
| | | alloy be used for construction. |
| 204 | Cool and Condense | Effluent gas from the reactor is cooled with a graphite block |
| | | or graphite tube heat exchanger. The condensate has both a |
| | | concentrated HCl aqueous phase and an organic phase. |
| 206 | Phase Separate | Gravity separation of the aqueous and organic phases from |
| | | Unit 204 is preferably achieved with a horizontal tank |
| | | provided with internal baffles to allow the heavy phase (most |
| | | likely the aqueous/acid phase, but the nature of the phases |
| | | depends on the exact composition of organics in the phases) to |
| | | be removed from one end of the vessel. The lighter phase |
| | | flows over the baffle into the second half of the vessel for |
| | | removal. The aqueous phase is then, in some embodiments, |
| | | stripped of organics. |
| 208 | HCl Recovery | The aqueous HCl stream from the separator is recovered as |
| | | anhydrous HCl for recycle to the reactor using traditionally |
| | | deployed approaches that are apparent to those of skill. |
| 210 | Product Split | A separation column, with refrigerated condensers at the top |
| | | to allow separation of the lights for recycle from the |
| | | chlorinated organics, is preferably used for this splitting |
| | | operation. |
| 212 | C2 Absorption and | Recovery of ethane and ethylene in the purge stream is |
| | Stripper | achieved by absorption into a hydrocarbon or other absorbing |
| | | liquid in an absorber, with a stripping operation in a second |
| | | column. The recovered hydrocarbons are then recycled |
| | | “back” to the main recycle stream and further to the reactor |
| | | 202. |
| 214 | Vent Treatment (TOX) | Vent treatment is achieved with an incinerator for the |
| | | oxidation of organics (including chlorinated organics) to water |
| | | vapor, carbon dioxide, and hydrogen chloride. The vent gas is |
| | | scrubbed with water to recover HCl as a relatively dilute (10 |
| | | to 20% HCl stream) for other uses. This unit is typical of |
| | | those found throughout the chemical industry and should be |
| | | apparent to those of skill. |
| 216 | Drying | Prior to the final separation of the VCM from the other |
| | | products in the raw product stream after lights have been |
| | | stripped, water is removed in a drying column. The pressure |
| | | and temperature are preferably adjusted such that the water is |
| | | removed from the bottom of the column and the dry product is |
| | | removed from the top. |
| 218 | VCM Columns | VCM is purified by methods as practiced in industry and |
| | | apparent to those of skill. |
| 220 | Heavies Column | Heavies are separated using a distillation column effecting the |
| | | separation of (a) the cis and trans 1,2 dichloroethylenes and |
| | | (b) EDC from heavier (higher molecular weight) components. |
| 282 | LT Reactor | The LT reactor is preferably a low temperature fluid bed |
| | | ethylene oxychlorination reactor providing a vertically |
| | | oriented reactor system with gas feed at the bottom and an |
| | | outlet at the top. Vertical cooling tubes are disposed in the |
| | | bed and internal cyclones (up to 3 in series) are located at the |
| | | top. Typical diameter is less than 20 feet. The height of the |
| | | fluid bed is between 30 and 50 feet, with total height of 80 feet |
| | | for the reactor. A fixed-bed system is used in an alternative |
| | | embodiment. |
| 286 | Residual HCl —Cool | Remaining HCl from the LT reactor is scrubbed and |
| | and Scrub | neutralized for disposal. This uses a system as is practiced by |
| | | those of skill for the operation following standard |
| | | oxychlorination reactors. |
| 293 | Furnaces | These are high temperature gas fired furnaces for the cracking |
| | | of EDC to VCM. The EDC is vaporized and passes through |
| | | the tubes within the furnace at temperatures of approximately |
| | | 600° C. to convert a portion of the EDC to VCM and HCl. This |
| | | is typical of furnaces used in industry today. |
| 295 | VCM Finishing and | VCM finishing and HCl recovery are achieved with a quench |
| | HCl Recovery | column or drum and separation columns as used in industry |
| | | today for the recovery of unconverted EDO, recovery and |
| | | recycle of the HCl, and purification of the VCM product. |
| 296 | Second Stage Reactor | This is a visioned secondary reactor for reacting remaining |
| | | HCl to near completion. In alternative envisioned contexts, |
| | | this reactor is either fixed or fluid bed; and in some envisioned |
| | | embodiments, it incorporates a standard commercially |
| | | available oxychlorination catalyst. |
| 298 | Res. HCl | With essentially complete conversion of HCl in the reactor, |
| | Neutralization | the recovery of the residual is not justified. The aqueous |
| | | solution is neutralized with any available alkaline material |
| | | (caustic, calcium hydroxide, calcium carbonate, ammonia, |
| | | etc.). The effluent is then sent to waste treatment. This |
| | | process would most likely be done in a closed tank, possibly |
| | | with an agitator. Depending on the amount of residual HCl, |
| | | cooling may need to be provided by a recirculation stream. |
| 502 | Reactor | Ethylene/ethane oxydehydro-chlorination reactor. |
| | | A fluid bed version (preferred) of the reactor is a vertically |
| | | oriented reactor system with gas feed at bottom and with the |
| | | outlet at the top. Vertical cooling tubes are positioned in the |
| | | bed, and internal cyclones (up to 3 in series) are located at the |
| | | top. Typical diameter of the reactor is less than 20 feet. |
| | | Height of fluid bed is between 30 feet and 50 feet, with a total |
| | | height of 80 feet for the reactor. |
| | | The fixed bed version of the reactor is a vertical exchanger |
| | | type catalytic reactor with tubes from 1 to 1.5 inches. The |
| | | reactor temperature of >400° C. requires that a high nickel |
| | | alloy be used for construction. |
| 510 | Cool and Condense | Effluent gas from the reactor is cooled with a graphite block, |
| | | graphite tube or other suitable heat exchanger. The condensate |
| | | has both a concentrated HCl aqueous phase and an organic |
| | | phase. |
| 516 | Phase Separate | Gravity separation of the aqueous and organic phases from |
| | | Step 510 is preferably achieved with a horizontal tank |
| | | provided with internal baffles to allow the heavy phase (most |
| | | likely the aqueous/acid phase, but the nature of the phases |
| | | depend on the exact composition of organics in the phases) to |
| | | be removed from one end of the vessel. The lighter phase |
| | | flows over the baffle into the second half of the vessel for |
| | | removal. The aqueous phase is then, in some embodiments, |
| | | stripped of organics. |
| 518 | Product Split | A separation column, with refrigerated condensers at the top |
| | | to allow separation of the lights for recycle from the |
| | | chlorinated organics, is preferably used for this splitting |
| | | operation. |
| 520 | Drying | Prior to the final separation of the VCM from the other |
| | | products in the raw product stream after lights have been |
| | | stripped, water is removed in a drying column. The pressure |
| | | and temperature are preferably adjusted such that the water is |
| | | removed from the bottom of the column and the dry product is |
| | | removed from the top. |
| 522 | VCM Purification | VCM is purified by methods as practiced in industry and |
| | | apparent to those of skill. |
| 524 | EDC Purification | Heavies are separated using a distillation column effecting the |
| | | separation of (a) the cis and trans 1,2 dichloroethylenes and |
| | | (b) EDC from heavier (higher molecular weight) components. |
| 538 | HCl Recovery | The aqueous HCl stream from the separator is recovered as |
| | | anhydrous HCl for recycle to the reactor using traditionally |
| | | deployed approaches which are apparent to those of skill. |
| 570 | HCl Absorption | Recovery of HCl by water absorption. |
| 580 | Vent Treatment | Vent treatment to make effluent safe for environmental |
| | | release. This can be achieved with an incinerator for the |
| | | oxidation of organics (including chlorinated organics) to water |
| | | vapor, carbon dioxide, and hydrogen chloride. The vent gas is |
| | | scrubbed with water to recover HCl as a relatively dilute (10 |
| | | to 20% HCl stream) for other uses. This unit is typical of |
| | | those found throughout the chemical industry and should be |
| | | apparent to those of skill. The unit may also be a catalytic |
| | | oxidizer or other flame-less oxidation apparatus. |
| 590 | Hydrogenation | Cis- and trans-1,2-dichloroethylene are hydrogenated to yield |
| | | EDC. The hydrogenation may be afforded in either the gas- or |
| | | liquid-phase. Liquid-phase hydrogenation would occur in |
| | | suitable solvent. EDC may be used as suitable solvent. The |
| | | reaction could occur in any suitable reactor with either fixed |
| | | or suspended catalyst. The catalyst may be either |
| | | heterogeneous or homogeneous. An adiabatic reactor packed |
| | | with suitable heterogeneous catalyst using dilution with EDC |
| | | to control reactor temperature may be employed. |
| 602 | Reactor | Ethylene/ethane oxydehydro-chlorination reactor. |
| | | A fluid bed version (preferred) of the reactor is a vertically |
| | | oriented reactor system with gas feed at bottom and with the |
| | | outlet at the top. Vertical cooling tubes are positioned in the |
| | | bed, and internal cyclones (up to 3 in series) are located at the |
| | | top. Typical diameter of the reactor is less than 20 feet. |
| | | Height of fluid bed is between 30 feet and 50 feet, with a total |
| | | height of 80 feet for the reactor. |
| | | The fixed bed version of the reactor is a vertical exchanger |
| | | type catalytic reactor with tubes from 1 to 1.5 inches. The |
| | | reactor temperature of >400° C. requires that a high nickel |
| | | alloy be used for construction. |
| 610 | Cool and Condense | Effluent gas from the reactor is cooled with a graphite block, |
| | | graphite tube or other suitable heat exchanger. The condensate |
| | | has both a concentrated HCl aqueous phase and an organic |
| | | phase. |
| 616 | Phase Separate | Gravity separation of the aqueous and organic phases from |
| | | Step 610 is preferably achieved with a horizontal tank |
| | | provided with internal baffles to allow the heavy phase (most |
| | | likely the aqueous/acid phase, but the nature of the phases |
| | | depend on the exact composition of organics in the phases) to |
| | | be removed from one end of the vessel. The lighter phase |
| | | flows over the baffle into the second half of the vessel for |
| | | removal. The aqueous phase is then, in some embodiments, |
| | | stripped of organics. |
| 618 | Product Split | A separation column, with refrigerated condensers at the top |
| | | to allow separation of the lights for recycle from the |
| | | chlorinated organics, is preferably used for this splitting |
| | | operation. |
| 620 | Drying | Prior to the final separation of the VCM from the other |
| | | products in the raw product stream after lights have been |
| | | stripped, water is removed in a drying column. The pressure |
| | | and temperature are preferably adjusted such that the water is |
| | | removed from the bottom of the column and the dry product is |
| | | removed from the top. |
| 622 | VCM Purification | VCM is purified by methods as practiced in industry and |
| | | apparent to those of skill. |
| 624 | EDC Purification | Heavies are separated using a distillation column effecting the |
| | | separation of (a) the cis and trans 1,2 dichloroethylenes and |
| | | (b) EDC from heavier (higher molecular weight) components. |
| 626 | Hydrogenation | Cis- and trans-1,2-dichloroethylene are hydrogenated to yield |
| | | EDC. The hydrogenation may be afforded in either the gas- or |
| | | liquid-phase. Liquid-phase hydrogenation would occur in |
| | | suitable solvent. EDC may be used as suitable solvent. The |
| | | reaction could occur in any suitable reactor with either fixed |
| | | or suspended catalyst. The catalyst may be either |
| | | heterogeneous or homogeneous. An adiabatic reactor packed |
| | | with suitable heterogeneous catalyst using dilution with EDC |
| | | to control reactor temperature may be employed. |
| 638 | HCl Vaporization | The aqueous HCl stream from the separator is vaporized to |
| | | form suitable feed for reactor 642. |
| 642 | Conventional | A reactor system suitable for conversion of ethylene, HCl and |
| | Oxychlorination | oxygen to EDC. |
| 670 | HCl Absorption | Recovery of HCl by water absorption. |
| 675 | C2 Absorption and | Recovery of ethane and ethylene in the purge stream is |
| | Stripper | achieved by absorption into a hydrocarbon or other absorbing |
| | | liquid in an absorber, with a stripping operation in a second |
| | | column. The recovered hydrocarbons are then recycled |
| | | “back” to the main recycle stream and further to the reactor. |
| 680 | Vent Treatment | Vent treatment to make effluent safe for environmental |
| | | release. This can be achieved with an incinerator for the |
| | | oxidation of organics (including chlorinated organics) to water |
| | | vapor, carbon dioxide, and hydrogen chloride. The vent gas is |
| | | scrubbed with water to recover HCl as a relatively dilute (10 |
| | | to 20% HCl stream) for other uses. This unit is typical of |
| | | those found throughout the chemical industry and should be |
| | | apparent to those of skill. The unit may also be a catalytic |
| | | oxidizer or other flameless oxidation apparatus. |
| 690 | Kellogg Process | Reactor for the conversion of ethylene, the oxygen contained |
| | | in air and aqueous HCl to EDC. The reaction takes place in |
| | | aqueous media. |
| 692 | Vaporization | Suitable unit for the vaporization of EDC and EtCl. |
| 694 | Stripper | Suitable unit for the steam stripping of organic components |
| | | from aqueous reaction media. |

### EXAMPLES

Specifics in catalysts are further clarified by a consideration of the following examples, which are intended to be purely exemplary.

### Example 1

To demonstrate the production of vinyl chloride from a stream comprising ethylene, a porous, refractory composition comprising lanthanum was prepared. A solution of LaCl₃ in water was prepared by dissolving one part of commercially available hydrated lanthanum chloride (obtained from J. T. Baker Chemical Company) in 8 parts of deionized water. Dropwise addition with stirring of ammonium hydroxide (obtained from Fisher Scientific, certified ACS specification) to neutral pH (by universal test paper) caused the formation of a gel. The mixture was centrifuged, and the solution decanted away from the solid. Approximately 150 ml of deionized water was added and the gel was stirred vigorously to disperse the solid. The resulting solution was centrifuged and the solution decanted away. This washing step was repeated two additional times. The collected, washed gel was dried for two hours at 120 degrees Celsius and subsequently calcined at 550 deg. C. for four hours in air. The resulting solid was crushed and sieved to yield particles suitable for additional testing. This procedure produced a solid matching the X-ray powder diffraction pattern of LaOCl.

The particles were placed in a pure nickel (alloy 200) reactor. The reactor was configured such that ethylene, ethane, HCl, O₂ and inert gas (He and Ar mixture) could be fed to the reactor. The function of the argon was as an internal standard for the analysis of the reactor feed and effluent by gas chromatography. Space time is calculated as the volume of catalyst divided by the flow rate at standard conditions. Feed rates are molar ratios. The reactor system was immediately fed an ethane-containing stream with the stoichiometry of one ethane, one HCl and one oxygen. This provides balanced stoichiometry for the production of VCM from ethylene.

Table 3 below sets forth the results of reactor testing using this composition.

Column 1 of Table 3 shows the high selectivity to vinyl chloride when the catalyst system is fed ethylene under oxidizing conditions in the presence of HCl. The composition contains helium in order to mimic a reactor operated with air as the oxidant gas.

Column 2 of Table 3 shows the high selectivity to vinyl chloride when the catalyst system is fed ethylene under oxidizing conditions in the presence of HCl. The composition is now fuel rich to avoid limitations imposed by flammability and contains no helium.

Column 3 of Table 3 shows the high selectivity to vinyl chloride and ethylene when the catalyst system is fed ethane under oxidizing conditions in the presence of HCl. The composition mimics a reactor operated with air as the oxidant gas. There is no ethylene present in the feed. The ethylene present in the reactor is the product of the partial oxidation of ethane.

Column 4 of Table 3 shows the result when both ethane and ethylene are fed. The reactor is operated in such a way as to insure that the amount of ethylene entering the reactor and exiting the reactor are equal. Operated in this fashion, the ethylene gives the appearance of an inert diluent, and only ethane is being converted. The results show a high yield of vinyl chloride and 1,2-dichloroethane. Argon is used as an internal standard to insure that the ethylene flux entering the reactor and the ethylene flux exiting the reactor are equal. The ratio of the ethylene to argon integrated chromatographic peak is identical for the reactor feed and product stream. In this way the recycle of ethylene is simulated within the reactor device.

TABLE 3
Feed Mole Ratios

| | | | | |
|---|---|---|---|---|
| C₂H₄ | 2 | 3.7 | 0 | 3 |
| C₂H₆ | 0 | 0 | 1 | 2 |
| HCl | 2 | 2 | 1 | 2.5 |
| O₂ | 1 | 1 | 1 | 1 |
| Inerts | 6.8 | 0 | 4 | 0 |
| T (deg. C.) | 401 | 400 | 401 | 419 |
| Space time (s) | 12.3 | 5.0 | 21.8 | 12.4 |
| O₂ conv. (pct) | 47.3 | 53.7 | 54.8 | 93.9 |

Selectivities (Percent)

| | | | | |
|---|---|---|---|---|
| C₂H₄ | — | — | 44.7 | — |
| C₂H₄Cl₂ | 10.7 | 14.0 | 0.1 | 12.8 |
| VCM | 76.6 | 78.1 | 34.5 | 68.5 |

### Example 2

To further demonstrate the utility of the composition, ethylene is oxidatively converted to vinyl chloride using a variety of chlorine sources. A solution of LaCl₃ in water was prepared by dissolving one part of commercially available hydrated lanthanum chloride (purchased from Avocado Research Chemicals Ltd.) in 6.6 parts of deionized water. Rapid addition with stirring of 6 M ammonium hydroxide in water (diluted certified ACS reagent, obtained from Fisher Scientific) caused the formation of a gel. The mixture was filtered to collect the solid. The collected gel was dried at 120 deg C. prior to calcination at 550 deg C. for four hours in air. The resulting solid was crushed and sieved. The sieved particles were placed in a pure nickel (alloy 200) reactor. The reactor was configured such that ethylene, HCl, oxygen, 1,2-dichloroethane, carbon tetrachloride and helium could be fed to the reactor. Space time is calculated as the volume of catalyst divided by the flow rate at standard temperature and pressure. Feed rates are molar ratios. The composition was heated to 400 deg C. and treated with a 1:1:3 HCl:O₂:He mixture for 2 hours prior to the start of operation.

The composition formed was operated to produce vinyl chloride by feeding ethylene, a chlorine source and oxygen at 400 deg C. The following table shows data obtained between 82 and 163 hours on stream using different chlorine sources. Chlorine is supplied as HCl, carbon tetrachloride and 1,2-dichloroethane. VCM signifies vinyl chloride. Space time is calculated as the volume of catalyst divided by the flow rate at standard temperature and pressure. The reactors are operated with the reactor exit at ambient pressure. Both ethylene and 1,2-dichloroethane are termed to be C2 species.

TABLE 4
Feed mole ratios

| | | | | |
|---|---|---|---|---|
| C₂H₄ | 2.0 | 2.0 | 2.0 | 2.0 |
| C₂H₆ | 0.0 | 0.0 | 0.0 | 0.0 |
| CCl₄ | 0.5 | 0.5 | 0.0 | 0.0 |
| C₂H₄Cl₂ | 0.0 | 0.0 | 1.8 | 0.0 |
| HCl | 0.0 | 0.0 | 0.0 | 1.9 |
| O₂ | 1.0 | 1.0 | 1.0 | 1.0 |
| He + Ar | 8.9 | 9.0 | 8.9 | 6.7 |
| T (deg C.) | 400 | 399 | 401 | 400 |
| Space time (s) | 8.0 | 4.0 | 8.6 | 4.9 |

Fractional conversions (Percent)

| | | | | |
|---|---|---|---|---|
| C₂H₄ | 40.4 | 27.0 | 18.7 | 20.1 |
| C₂H₆ | 0.0 | 0.0 | 0.0 | 0.0 |
| CCl₄ | 94.8 | 78.4 | 0.0 | 0.0 |
| C₂H₄Cl₂ | 0.0 | 0.0 | 98.3 | 0.0 |
| HCl | 0.0 | 0.0 | 0.0 | 44.7 |
| O₂ | 68.8 | 42.0 | 55.2 | 37.8 |

Selectivities based on moles of C₂ converted

| | | | | |
|---|---|---|---|---|
| VCM | 59.6 | 56.4 | 86.0 | 78.5 |
| C₂H₄Cl₂ | 14.8 | 30.7 | 0.0 | 2.2 |
| C₂H₅Cl | 0.6 | 0.4 | 0.2 | 1.6 |

These data show that a variety of chlorine sources can be used in the oxidative production of vinyl. The use of carbon tetrachloride, 1,2-dichloroethane and HCl all produce vinyl chloride as the dominant product.

### Example 3

A solution of LaCl₃ in water was prepared by dissolving one part of commercially available hydrated lanthanum chloride (purchased from Avocado Research Chemicals Ltd.) in 6.67 parts of deionized water. Rapid addition with stirring of 6 M ammonium hydroxide in water (diluted certified ACS reagent, obtained from Fisher Scientific) caused the formation of a gel and yielded a final pH of 8.85. The mixture was filtered to collect the solid. The collected material was calcined in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor.

Table 5 shows data wherein the reactor feeds were adjusted such that the flux of ethylene (moles/minute) entering the reactor and the flux of ethylene exiting the reactor were substantially equal. Reactor feeds were similarly adjusted such that the fluxes of HCl entering and exiting the reactor were substantially equal. Oxygen conversion was set at slightly less than complete conversion to enable the monitoring of catalyst activity. Operated in this manner, the consumed feeds are ethane, oxygen, and chlorine. Both ethylene and HCl give the appearance of neither being created nor consumed. Space time is calculated as the volume of catalyst divided by the flow rate at standard temperature and pressure. The example further illustrates the use of chlorine gas as a chlorine source in the production of vinyl chloride.

TABLE 5
Feed mole ratios

| | |
|---|---|
| C₂H₄ | 2.1 |
| C₂H₈ | 4.5 |
| Cl₂ | 0.5 |
| HCl | 2.4 |
| O₂ | 1.0 |
| He + Ar | 7.4 |
| T (° C.) | 400 |
| Space time (s) | 9.4 |

Fractional conversions (Pct.)

| | |
|---|---|
| C₂H₄ | 1.8 |
| C₂H₆ | 27.3 |
| Cl₂ | 99.8 |
| HCl | −1.4 |
| O₂ | 96.4 |

Selectivities (Pct)

| | |
|---|---|
| VCM | 79.0 |
| C₂H₄Cl₂ | 7.2 |
| C₂H₅Cl | 1.7 |
| COₓ | 5.1 |
| C₂H₄ | 0.5 |

In common with all examples herein, VCM signifies vinyl chloride. C₂H₄Cl₂ is solely 1,2-dichloroethane. COX is the combination of CO and CO₂.

### Example 4 Through Example 11

Example 4 through Example 11 illustrate the preparation of numerous rare earth compositions, each containing only one rare earth material. Data illustrating the performance of these compositions are set forth in Table 6.

### Example 4

A solution of LaCl₃ in water was prepared by dissolving one part of commercially available hydrated lanthanum chloride (purchased from Aldrich Chemical Company) in 6.67 parts of deionized water. Rapid addition with stirring of 6 M ammonium hydroxide in water (diluted certified ACS reagent, obtained from Fisher Scientific) caused the formation of a gel. The mixture was centrifuged to collect the solid. Solution was decanted away from the gel and discarded. The gel was re-suspended in 6.66 parts of deionized water. Centrifuging allowed collection of the gel. The collected gel was dried at 120 deg C. prior to calcination at 550 deg C. for four hours in air. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. Powder x-ray diffraction shows the material to be LaOCl. The BET surface area is measured to be 42.06 m²/g. The specific performance data for this example are set forth below in Table 6.

### Example 5

A solution of NdCl₃ in water was prepared by dissolving one part of commercially available hydrated neodymium chloride (Alfa Aesar) in 6.67 parts of deionized water. Rapid addition with stirring of 6 M ammonium hydroxide in water (diluted certified ACS reagent, obtained from Fisher Scientific) caused the formation of a gel. The mixture was filtered to collect the solid. The collected gel was dried at 120 deg C. prior to calcination in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. Powder x-ray diffraction shows the material to be NdOCl. The BET surface area is measured to be 22.71 m²/g. The specific performance data for this example are set forth below in Table 6.

### Example 6

A solution of PrCl₃ in water was prepared by dissolving one part of commercially available hydrated praseodymium chloride (Alfa Aesar) in 6.67 parts of deionized water. Rapid addition with stirring of 6 M ammonium hydroxide in water (diluted certified ACS reagent, obtained from Fisher Scientific) caused the formation of a gel. The mixture was filtered to collect the solid. The collected gel was dried at 120 deg C. prior to calcination in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. Powder x-ray diffraction shows the material to be PrOCl. The BET surface area is measured to be 21.37 m²/g. The specific performance data for this example are set forth below in Table 6.

### Example 7

A solution of SmCl₃ in water was prepared by dissolving one part of commercially available hydrated samarium chloride (Alfa Aesar) in 6.67 parts of deionized water. Rapid addition with stirring of 6 M ammonium hydroxide in water (diluted certified ACS reagent, obtained from Fisher Scientific) caused the formation of a gel. The mixture was filtered to collect the solid. The collected gel was dried at 120 deg C. prior to calcination at 500 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. Powder x-ray diffraction shows the material to be SmOCl. The BET surface area is measured to be 30.09 m²/g. The specific performance data for this example are set forth below in Table 6.

### Example 8

A solution of HoCl₃ in water was prepared by dissolving one part of commercially available hydrated holmium chloride (Alfa Aesar) in 6.67 parts of deionized water. Rapid addition with stirring of 6 M ammonium hydroxide in water (diluted certified ACS reagent, obtained from Fisher Scientific) caused the formation of a gel. The mixture was filtered to collect the solid. The collected gel was dried at 120 deg C. prior to calcination at 500 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 20.92 m²/g. The specific performance data for this example are set forth below in Table 6.

### Example 9

A solution of ErCl₃ in water was prepared by dissolving one part of commercially available hydrated erbium chloride (Alfa Aesar) in 6.67 parts of deionized water. Rapid addition with stirring of 6 M ammonium hydroxide in water (diluted certified ACS reagent, obtained from Fisher Scientific) caused the formation of a gel. The mixture was filtered to collect the solid. The collected gel was dried at 120 deg C. prior to calcination at 500 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 19.80 m²/g. The specific performance data for this example are set forth below in Table 6.

### Example 10

A solution of YbCl₃ in water was prepared by dissolving one part of commercially available hydrated ytterbium chloride (Alfa Aesar) in 6.67 parts of deionized water. Rapid addition with stirring of 6 M ammonium hydroxide in water (diluted certified ACS reagent, obtained from Fisher Scientific) caused the formation of a gel. The mixture was filtered to collect the solid. The collected gel was dried at 120 deg C. prior to calcination at 500 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 2.23 m²/g. The specific performance data for this example are set forth below in Table 6.

### Example 11

A solution of YCl₃ in water was prepared by dissolving one part of commercially available hydrated yttrium chloride (Alfa Aesar) in 6.67 parts of deionized water. Rapid addition with stirring of 6 M ammonium hydroxide in water (diluted certified ACS reagent, obtained from Fisher Scientific) caused the formation of a gel. The mixture was filtered to collect the solid. The collected gel was dried at 120 deg C. prior to calcination at 500 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 29.72 m²/g. The specific performance data for this example are set forth below in Table 6.

TABLE 6
Rare Earth Oxychloride Compositions
Operated to Produce Vinyl Chloride

| Example | | | | | | | |
|---|---|---|---|---|---|---|---|
| 5 | 6 | 7 | 8 | 9 | 10 | 11 | 12 |

Feed mole ratios

| | | | | | | | | |
|---|---|---|---|---|---|---|---|---|
| C₂H₄ | 3.6 | 4.2 | 3.7 | 3.6 | 3.6 | 3.6 | 4.2 | 3.6 |
| HCl | 2.0 | 2.3 | 2.0 | 2.0 | 2.0 | 2.0 | 2.3 | 2.0 |
| O₂ | 1.0 | 1.0 | 1.0 | 1.0 | 1.0 | 1.0 | 1.0 | 1.0 |
| He + Ar | 0.2 | 0.2 | 0.2 | 0.2 | 0.2 | 0.2 | 0.2 | 0.2 |
| T (deg C.) | 399 | 403 | 401 | 400 | 400 | 400 | 400 | 399 |
| Space time (s) | 8.7 | 21.3 | 11.4 | 17.6 | 17.7 | 22.8 | 23.1 | 21.3 |

Fractional conversions (Percent)

| | | | | | | | | |
|---|---|---|---|---|---|---|---|---|
| C₂H₄ | 23.7 | 13.2 | 22.8 | 14.7 | 12.7 | 15.4 | 3.3 | 13.8 |
| HCl | 47.6 | 24.9 | 40.9 | 20.8 | 15.9 | 22.4 | 5.0 | 19.8 |
| O₂ | 58.8 | 59.4 | 55.0 | 53.4 | 48.1 | 48.8 | 21.2 | 47.8 |

Selectivities (Percent)

| | | | | | | | | |
|---|---|---|---|---|---|---|---|---|
| VCM | 75.3 | 74.4 | 74.2 | 61.0 | 33.3 | 44.0 | 6.1 | 35.0 |
| C₂H₄Cl₂ | 11.3 | 2.9 | 6.1 | 2.9 | 14.5 | 17.5 | 8.8 | 18.8 |
| C₂H₅Cl | 3.5 | 6.9 | 4.4 | 10.6 | 16.8 | 12.8 | 37.0 | 16.5 |
| COₓ | 4.8 | 11.8 | 9.7 | 22.4 | 33.8 | 23.1 | 26.4 | 27.5 |

These data show the utility of bulk rare earth containing compositions for the conversion of ethylene containing streams to vinyl chloride.

### Example 12 Through Example 16

Example 12 through Example 16 illustrate the preparation of numerous rare earth compositions, each containing a mixture of rare earth materials. Data illustrating the performance of these data are set forth in Table 7.

### Example 12

A solution of LaCl₃ and NdCl₃ in water was prepared by dissolving one part of commercially available hydrated lanthanum chloride (purchased from Spectrum Quality Products) and 0.67 parts of commercially available hydrated neodymium chloride (Alfa Aesar) in 13.33 parts of deionized water. Rapid addition with stirring of 6 M ammonium hydroxide in water (diluted certified ACS reagent, obtained from Fisher Scientific) caused the formation of a gel. The final pH was measured as 8.96. The mixture was centrifuged to collect the solid. Solution was decanted away from the gel and discarded. The collected gel was dried at 80 deg C. prior to calcination in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 21.40 m²/g. The specific performance data for this example are set forth below in Table 7.

### Example 13

A solution of LaCl₃ and SmCl₃ in water was prepared by dissolving one part of commercially available hydrated lanthanum chloride (purchased from Spectrum Quality Products) and 0.67 parts of commercially available hydrated samarium chloride (Alfa Aesar) in 13.33 parts of deionized water. Rapid addition with stirring of 6 M ammonium hydroxide in water (diluted certified ACS reagent, obtained from Fisher Scientific) caused the formation of a gel. The final pH was measured as 8.96. The mixture was centrifuged to collect the solid. Solution was decanted away from the gel and discarded. The collected gel was dried at 80 deg C. prior to calcination in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 21.01 m²/g. The specific performance data for this example are set forth below in Table 7.

### Example 14

A solution of LaCl₃ and YCl₃ in water was prepared by dissolving one part of commercially available hydrated lanthanum chloride (purchased from Spectrum Quality Products) and 0.52 parts of commercially available hydrated yttrium chloride (Alfa Aesar) in 13.33 parts of deionized water. Rapid addition with stirring of 6 M ammonium hydroxide in water (diluted certified ACS reagent, obtained from Fisher Scientific) caused the formation of a gel. The final pH was measured as 8.96. The mixture was centrifuged to collect the solid. Solution was decanted away from the gel and discarded. The collected gel was dried at 80 deg C. prior to calcination in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 20.98 m²/g. The specific performance data for this example are set forth below in Table 7.

### Example 15

A solution of LaCl₃ and HoCl₃ in water was prepared by dissolving one part of commercially available hydrated lanthanum chloride (purchased from Spectrum Quality Products) and one part of commercially available hydrated holmium chloride (Alfa Aesar) in 13.33 parts of deionized water. Rapid addition with stirring of 6 M ammonium hydroxide in water (diluted certified ACS reagent, obtained from Fisher Scientific) caused the formation of a gel. The final pH was measured as 8.64. The mixture was centrifuged to collect the solid. Solution was decanted away from the gel and discarded. The collected gel was dried at 80 deg C. prior to calcination in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 19.68 m²/g. The specific performance data for this example are set forth below in Table 7.

### Example 16

A solution of LaCl₃ and HoCl₃ in water was prepared by dissolving one part of commercially available hydrated lanthanum chloride (purchased from Spectrum Quality Products) and 0.75 parts of commercially available hydrated ytterbium chloride (Alfa Aesar) in 13.33 parts of deionized water. Rapid addition with stirring of 6 M ammonium hydroxide in water (diluted certified ACS reagent, obtained from Fisher Scientific) caused the formation of a gel. The final pH was measured as 9.10. The mixture was centrifuged to collect the solid. Solution was decanted away from the gel and discarded. The collected gel was dried at 80 deg C. prior to calcination in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 20.98 m²/g. The specific performance data for this example are set forth below in Table 7.

TABLE 7
Performance of Compositions Containing
Two Rare earth materials

| Example | | | | |
|---|---|---|---|---|
| 13 | 14 | 15 | 16 | 17 |

Feed mole ratios

| | | | | | |
|---|---|---|---|---|---|
| C₂H₄ | 3.7 | 3.6 | 3.6 | 3.6 | 3.6 |
| HCl | 2.0 | 2.0 | 2.0 | 2.0 | 2.0 |
| O₂ | 1.0 | 1.0 | 1.0 | 1.0 | 1.0 |
| He + Ar | 0.2 | 0.2 | 0.2 | 0.2 | 0.2 |
| T (° C.) | 401 | 401 | 400 | 399 | 400 |
| Space time (s) | 3.7 | 15.7 | 13.7 | 16.9 | 20.6 |

Fractional conversions (Percent)

| | | | | | |
|---|---|---|---|---|---|
| C₂H₄ | 16.8% | 11.3 | 12.5 | 12.4 | 9.2 |
| HCl | 36.0 | 13.1 | 18.1 | 11.9 | 15.9 |
| O₂ | 45.9 | 47.2 | 52.2 | 47.1 | 38.7 |

Selectivities (Percent)

| | | | | | |
|---|---|---|---|---|---|
| VCM | 75.8 | 51.0 | 51.4 | 28.9 | 11.1 |
| C₂H₄Cl₂ | 9.7 | 7.5 | 12.4 | 14.5 | 20.6 |
| C₂H₅Cl | 4.1 | 11.8 | 8.9 | 17.0 | 23.8 |
| COₓ | 6.9 | 27.5 | 25.8 | 38.9 | 43.8 |

These data further show the utility of bulk rare earth containing compositions containing mixtures of the rare earth materials for the conversion of ethylene containing streams to vinyl chloride.

### Example 17 Through Example 24

Example 17 through Example 24 are compositions containing rare earth materials with other additives present.

### Example 17

A solution of LaCl₃ in water was prepared by dissolving one part of commercially available hydrated lanthanum chloride (purchased from Aldrich Chemical Company) in 6.67 parts of deionized water. 0.48 parts of ammonium hydroxide (Fisher Scientific) was added to 0.35 parts of commercially prepared CeO₂ powder (Rhone-Poulenc). The lanthanum and cerium containing mixtures were added together with stirring to form a gel. The resulting gel containing mixture was filtered and the collected solid was calcined in air at 550 deg C. for 4 hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The specific performance data for this example are set forth below in Table 8.

### Example 18

A lanthanum containing composition prepared using the method of Example 5 was ground with a mortar and pestle to form a fine powder. One part of the ground powder was combined with 0.43 parts BaCl₂ powder and further ground using a mortar and pestle to form an intimate mixture. The lanthanum and barium containing mixture was pressed to form chunks. The chunks were calcined at 800 deg C. in air for 4 hours. The resulting material was placed in a pure nickel (alloy 200) reactor. The reactor was configured such that ethylene, ethane, HCl, oxygen, and inert (helium and argon mixture) could be fed to the reactor. The specific performance data for this example are set forth below in Table 8.

### Example 19

Dried Grace Davison Grade 57 silica was dried at 120 deg C. for 2 hours. A saturated solution of LaCl₃ in water was formed using commercially available hydrated lanthanum chloride. The dried silica was impregnated to the point of incipient wetness with the LaCl₃ solution. The impregnated silica was allowed to air dry for 2 days at ambient temperature. It was further dried at 120 deg C. for 1 hour. The resulting material was placed in a pure nickel (alloy 200) reactor. The reactor was configured such that ethylene, ethane, HCl, oxygen, and inert (helium and argon mixture) could be fed to the reactor. The specific performance data for this example are set forth below in Table 8.

### Example 20

A solution of LaCl₃ in water was prepared by dissolving one part of commercially available hydrated lanthanum chloride (purchased from Spectrum Quality Products) in 6.67 parts of deionized water. Rapid addition with stirring of 6 M ammonium hydroxide in water (diluted certified ACS reagent, obtained from Fisher Scientific) caused the formation of a gel. The mixture was centrifuged to collect the solid. Solution was decanted away from the gel and discarded. The gel was re-suspended in 12.5 parts of acetone (Fisher Scientific), centrifuged, and the liquid decanted away and discarded. The acetone washing step was repeated 4 additional times using 8.3 parts acetone. The gel was re-suspended in 12.5 parts acetone and 1.15 parts of hexamethyldisilizane (purchased from Aldrich Chemical Company) was added and the solution was stirred for one hour. The mixture was centrifuged to collect the gel. The collected gel was allowed to air dry at ambient temperature prior to calcination in air at 550 deg C. for four hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The BET surface area is measured to be 58.82 m²/g. The specific performance data for this example are set forth below in Table 8.

### Example 21

A solution of LaCl₃ in water was prepared by dissolving one part of commercially available hydrated lanthanum chloride (Alfa Aesar) and 0.043 parts of commercially available HfCl₄ (purchased from Acros Organics) in 10 parts of deionized water. Rapid addition with stirring of 6 M ammonium hydroxide in water (diluted certified ACS reagent, obtained from Fisher Scientific) caused the formation of a gel. The mixture was centrifuged to collect the solid. Solution was decanted away from the gel and discarded. The collected gel was dried at 80 deg C. overnight prior to calcination at 550 deg C. for 4 hours. The specific performance data for this example are set forth below in Table 8.

### Example 22

A solution of LaCl₃ in water was prepared by dissolving one part of commercially available hydrated lanthanum chloride (Alfa Aesar) and 0.086 parts of commercially available HfCl₄ (purchased from Acros Organics) in 10 parts of deionized water. Rapid addition with stirring of 6 M ammonium hydroxide in water (diluted certified ACS reagent, obtained from Fisher Scientific) caused the formation of a gel. The mixture was centrifuged to collect the solid. Solution was decanted away from the gel and discarded The collected gel was dried at 80 deg C. overnight prior to calcination at 550 deg C. for 4 hours. The specific performance data for this example are set forth below in Table 8.

### Example 23

A solution of LaCl₃ in water was prepared by dissolving one part of commercially available hydrated lanthanum chloride (Alfa Aesar) and 0.043 parts of commercially available ZrOCl₂ (purchased from Acros Organics) in 10 parts of deionized water. Rapid addition with stirring of 6 M ammonium hydroxide in water (diluted certified ACS reagent, obtained from Fisher Scientific) caused the formation of a gel. The mixture was centrifuged to collect the solid. Solution was decanted away from the gel and discarded. The gel was re-suspended in 6.67 parts deionized water and subsequently centrifuged. The solution was decanted away and discarded. The collected gel was calcined at 550 deg C. for 4 hours. The specific performance data for this example are set forth below in Table 8.

### Example 24

A solution of LaCl₃ in water was prepared by dissolving commercially available hydrated lanthanum chloride in deionized water to yield a 2.16 M solution. Commercially produced zirconium oxide (obtained from Engelhard) was dried at 350 deg C. overnight. One part of the zirconium oxide was impregnated with 0.4 parts of the LaCl₃ solution. The sample was dried in air at room temperature and then calcined in air at 550 deg C. for 4 hours. The resulting solid was crushed and sieved. The sieved particles were placed in a pure nickel (alloy 200) reactor. The reactor was configured such that ethylene, ethane, HCl, oxygen, and inert (helium and argon mixture) could be fed to the reactor. The specific performance data for this example are set forth below in Table 8.

TABLE 8
Rare Earth Compositions with Additional Components

| Example | | | | | | | |
|---|---|---|---|---|---|---|---|
| 18 | 19 | 20 | 21 | 22 | 23 | 24 | 25 |

Feed mole ratios

| | | | | | | | | |
|---|---|---|---|---|---|---|---|---|
| C₂H₄ | 3.7 | 3.6 | 3.7 | 3.7 | 3.7 | 3.7 | 3.6 | 3.7 |
| HCl | 2.0 | 2.0 | 2.0 | 2.0 | 2.0 | 2.0 | 2.0 | 2.0 |
| O₂ | 1.0 | 1.0 | 1.0 | 1.0 | 1.0 | 1.0 | 1.0 | 1.0 |
| He + Ar | 0.2 | 0.2 | 0.2 | 0.2 | 0.2 | 0.2 | 0.2 | 0.2 |
| T (° C.) | 400 | 401 | 400 | 399 | 401 | 400 | 400 | 401 |
| Space | 4.8 | 20.3 | 6.7 | 3.6 | 7.9 | 7.8 | 12.8 | 16.7 |
| time (s) | | | | | | | | |

Fractional conversions (Percent)

| | | | | | | | | |
|---|---|---|---|---|---|---|---|---|
| C₂H₄ | 18.2 | 11.7 | 14.1 | 24.6 | 18.5 | 16.5 | 18.7 | 15.2 |
| HCl | 34.6 | 22.1 | 24.4 | 57.1 | 40.9 | 38.2 | 35.2 | 21.1 |
| O₂ | 55.6 | 33.2 | 48.0 | 52.0 | 50.3 | 47.4 | 50.9 | 56.4 |

Selectivities (Percent)

| | | | | | | | | |
|---|---|---|---|---|---|---|---|---|
| VCM | 64.5 | 54.6 | 53.6 | 56.0 | 76.4 | 71.8 | 73.2 | 55.1 |
| C₂H₄Cl₂ | 11.5 | 15.2 | 10.0 | 31.4 | 9.6 | 12.7 | 5.2 | 7.3 |
| C₂H₅Cl | 5.0 | 10.0 | 7.4 | 2.9 | 4.0 | 4.9 | 4.9 | 12.4 |
| COₓ | 10.8 | 18.6 | 26.6 | 6.0 | 7.6 | 8.8 | 13.6 | 24.1 |

These data show the production of vinyl chloride from ethylene containing streams using lanthanum-based catalysts that contain other elements or are supported.

### Example 25 Through Example 30

Example 25 through Example 30 show some of the modifications possible to alter the preparation of useful rare earth compositions.

### Example 25

A solution of LaCl₃ in water was prepared by dissolving one part of commercially available hydrated lanthanum chloride (purchased from Spectrum Quality Products) in 10 parts of deionized water. Rapid addition with stirring of 6 M ammonium hydroxide in water (diluted certified ACS reagent, obtained from Fisher Scientific) caused the formation of a gel. The mixture was centrifuged to collect the solid. Solution was decanted away from the gel and discarded. A saturated solution of 0.61 parts benzyltriethylammonium chloride (purchased from Aldrich Chemical Company) in deionized water was prepared. The solution was added to the gel and stirred. The collected gel was calcined at 550 deg C. for 4 hours. The specific performance data for this example are set forth below in Table 9. This example illustrates the use of added ammonium salts to alter the preparation of rare earth compositions.

### Example 26

A solution of LaCl₃ in water was prepared by dissolving one part of commercially available hydrated lanthanum chloride (purchased from Spectrum Quality Products) in 10 parts of deionized water. Rapid addition with stirring of 6 M ammonium hydroxide in water (diluted certified ACS reagent, obtained from Fisher Scientific) caused the formation of a gel. The mixture was centrifuged to collect the solid. One part glacial acetic acid was added to the gel and the gel re-dissolved. Addition of the solution to 26 parts of acetone caused the formation of a precipitate. The solution was decanted away and the solid was calcined at 550 deg C. for 4 hours. The specific performance data for this example are set forth below in Table 9. This example shows the preparation of useful lanthanum compositions by the decomposition of carboxylic acid adducts of chlorine containing rare earth compounds.

### Example 27

A solution of LaCl₃ in water was prepared by dissolving one part of commercially available hydrated lanthanum chloride (purchased from Spectrum Quality Products) in 10 parts of deionized water. Rapid addition with stirring of 6 M ammonium hydroxide in water (diluted certified ACS reagent, obtained from Fisher Scientific) caused the formation of a gel. The mixture was centrifuged to collect the solid. The collected gel was re-suspended in 3.33 parts of deionized water. Subsequent addition of 0.0311 parts of phosphoric acid reagent (purchased from Fisher Scientific) produced no visible change in the suspended gel. The mixture was again centrifuged and the solution decanted away from the phosphorus containing gel. The collected gel was calcined for at 550 deg C. for 4 hours. The calcined solid had a BET surface area of 33.05 m²/g. The specific performance data for this example are set forth below in Table 9. This example shows the preparation of a rare earth composition also containing phosphorus, as phosphate.

### Example 28

A solution of LaCl₃ in water was prepared by dissolving one part of commercially available hydrated lanthanum chloride (purchased from Acros Organics) in 6.66 parts of deionized water. A solution was formed by mixing 0.95 parts of commercially available DABCO, or 1,4-diazabicyclo[2.2.2]octane, (purchased from ICN Pharmaceuticals) dissolved in 2.6 parts of deionized water. Rapid mixing with stirring of the two solutions caused the formation of a gel. The mixture was centrifuged to collect the solid. The collected gel was re-suspended in 6.67 parts of deionized water. The mixture was again centrifuged and the solution decanted away from the gel. The collected gel was calcined for 4 hours at 550 deg C. The calcined solid had a BET surface area of 38.77 m²/g. The specific performance data for this example are set forth below in Table 9. This example shows the utility of an alkyl amine in the preparation of a useful rare earth composition.

### Example 29

A solution of LaCl₃ in water was prepared by dissolving one part of commercially available hydrated lanthanum chloride (purchased from Acros Organics) in 10 parts of deionized water. To this solution, 2.9 parts of commercially available tetramethyl ammonium hydroxide (purchased from Aldrich Chemical Company) was added rapidly and with stirring, causing the formation of a gel. The mixture was centrifuged and the solution decanted away. The collected gel was resuspended in 6.67 parts of deionized water. The mixture was again centrifuged and the solution decanted away from the gel. The collected gel was calcined for 4 hours at 550 deg C. The calcined solid had a BET surface area of 80.35 m²/g. The specific performance data for this example are set forth below in Table 9. This example shows the utility of an alkyl ammonium hydroxide for formation of a useful rare earth composition.

### Example 30

A solution of LaCl₃ in water was prepared by dissolving one part of commercially available hydrated lanthanum chloride (purchased from Avocado Research Chemicals Ltd.) in 6.67 parts of deionized water. To this solution, 1.63 parts of commercially available 5 N NaOH solution (Fisher Scientific) was added rapidly and with stirring, causing the formation of a gel. The mixture was centrifuged and the solution 110 decanted away. The collected gel was calcined for 4 hours at 550 deg C. The calcined solid had a BET surface area of 16.23 m²/g. The specific performance data for this example are set forth below in Table 9. This example shows the utility of non-nitrogen containing bases for the formation of catalytically interesting materials. Although potentially functional the tested materials appear to be inferior to those produced using nitrogen containing bases.

TABLE 9
Additional Preparation Methods for
Lanthanum Containing Compositions

| Example | | | | |
|---|---|---|---|---|
| 26 | 27 | 28 | 29 | 30 |

Feed mole ratios

| | | | | | |
|---|---|---|---|---|---|
| C₂H₄ | 3.6 | 3.7 | 3.6 | 3.7 | 3.7 |
| HCl | 2.0 | 2.0 | 2.0 | 2.0 | 2.0 |
| O₂ | 1.0 | 1.0 | 1.0 | 1.0 | 1.0 |
| He + Ar | 0.2 | 0.2 | 0.2 | 0.2 | 0.2 |
| T (° C.) | 401 | 400 | 400 | 399 | 400 |
| Space | 8.6 | 20.8 | 4.7 | 8.7 | 6.2 |
| time (s) | | | | | |

Fractional conversions (Percent)

| | | | | | |
|---|---|---|---|---|---|
| C₂H₄ | 18.8 | 8.7 | 15.6 | 17.4 | 21.0 |
| HCl | 35.8 | 7.7 | 20.0 | 41.5 | 48.4 |
| O₂ | 53.0 | 32.6 | 48.8 | 50.6 | 56.8 |

Selectivities (Percent)

| | | | | | |
|---|---|---|---|---|---|
| VCM | 73.4 | 26.0 | 72.1 | 76.8 | 77.6 |
| C₂H₄Cl₂ | 8.7 | 11.9 | 7.1 | 7.3 | 7.8 |
| C₂H₅Cl | 3.5 | 22.7 | 5.6 | 4.2 | 2.9 |
| COₓ | 9.8 | 38.6 | 12.7 | 7.6 | 6.3 |

### Example 31

A solution of LaCl₃ in water was prepared by dissolving one part of commercially available hydrated lanthanum chloride (96% minimum purity; supplied by AMR) in 6.67 parts of deionized water. Rapid addition with stirring of 1.33 parts of 6 M ammonium hydroxide in water (diluted certified ACS reagent, obtained from Fisher Scientific) caused the formation of a gel. The mixture was centrifuged to collect the solid. Solution was decanted away from the gel and discarded. The gel was re-suspended in 6.67 parts of deionized water. Centrifuging allowed collection of the gel. The collected gel was dried at 80 deg C. prior to calcination at 550 deg C. for four hours in air. The resulting solid was crushed and sieved. Powder x-ray diffraction shows the material to be LaOCl. The BET surface area is measured to be 36.0006 m²/g. The sieved particles were placed in a pure nickel (alloy 200) reactor. The reactor was configured such that ethane, HCl, oxygen, and inert (helium and argon mixture) could be fed to the reactor. The reactor was operated at 400 deg C. at near ambient pressure. The feeds were adjusted to give an ethane: HCl: oxygen: inert ratio of 1:2:6.7:24.5. Feed rates were adjusted to give >99.6% ethane conversion. Molar carbon selectivities were as follows: vinyl chloride, 42.2 percent; dichloroethylenes, 34.8 percent; carbon monoxide, 14.9 percent; and carbon dioxide, 7.2 percent.

The present invention has been described in an illustrative manner. In this regard, it is evident that those skilled in the art, once given the benefit of the foregoing disclosure, may now make modifications to the specific embodiments described herein without departing from the spirit of the present invention. Such modifications are to be considered within the scope of the present invention which is limited solely by the scope and spirit of the appended claims.

What is claimed is:

1. A process for producing vinyl chloride comprising: (a) combining reactants including ethane, ethylene, or mixtures thereof with an oxygen source and a chlorine source in a reactor containing a suitable catalyst under fuel-lean process conditions sufficient to convert substantially all of the C2 hydrocarbon(s) fed and to produce a product stream comprising vinyl chloride and hydrogen chloride; and (b) recycling unreacted hydrogen chloride back for use in step (a).

2. The process of claim 1, wherein the oxygen is supplied as air.

3. The process of claim 2, wherein the oxygen is present at greater than three times the volumetric concentration of C2 hydrocarbons in the feed.

4. The process of claim 3 wherein greater than about 97 mole percent of the C2 hydrocarbon in the feed is converted, such that there is essentially no recoverable C2 hydrocarbon and no need to recycle C2 hydrocarbon back to step (a).

5. The process of claim 1, wherein the catalyst comprises one or more rare earth materials, with the proviso that the catalyst is substantially free of iron and copper and with the further proviso that when cerium is present the catalyst further comprises at least one more rare earth element other than cerium.

6. The method of claim 5 wherein the rare earth material component is selected from lanthanum, neodymium, praseodymium, and mixtures thereof.

7. The process of claim 1, wherein the chlorine source is a gas and comprises at least one of the following: hydrogen chloride, chlorine, a chlorinated hydrocarbon containing labile chlorines, and mixtures thereof.

8. The process of claim 1, wherein cis/trans-1,2-dichloroethylenes present in the product stream are hydrogenated with hydrogen to form ethylene dichloride (1,2-dichloroethane), which optionally is recycled, at least in part, to the reactor in step (a).

9. A process of manufacturing vinyl chloride comprising: (a) combining reactants including ethane, optionally ethylene, an oxygen source, and a chlorine source in a reactor containing a suitable catalyst under conditions sufficient to produce a product stream comprising vinyl chloride and hydrogen chloride; and (b) catalytically reacting said hydrogen chloride in a second reactor to provide a second reactor effluent essentially devoid of hydrogen chloride; and (c) recycling said second reactor effluent to catalytically react together with said ethane, said optional ethylene, said oxygen source, and said chlorine source in said combining step (a).

10. The process of claim 9, wherein the catalyst in step (a) comprises one or more rare earth materials, with the proviso that the catalyst is substantially free of iron and copper and with the further proviso that when cerium is present the catalyst further comprises at least one more rare earth element other than cerium.

11. The process of claim 10 wherein the rare earth material component is selected from lanthanum, neodymium, praseodymium, and mixtures thereof.

12. The process of claim 9, wherein the chlorine source is a gas and comprises at least one of the following: hydrogen chloride, chlorine, a chlorinated hydrocarbon containing labile chlorines, and mixtures thereof.

13. The process of claim 9 wherein the hydrogen chloride is catalytically reacted in step (b) in a conventional oxychlorination process to form ethyl chloride and/or ethylene dichloride.

14. The process of claim 9 wherein the hydrogen chloride is catalytically reacted in step (b) in an aqueous liquid phase oxychlorination process to form ethyl chloride and/or ethylene dichloride.

15. The process of claim 13 or 14 wherein the ethyl chloride and/or ethylene dichloride produced are recycled, at least in part, to oxydehydro-chlorination step (a).

16. The process of claim 9, wherein cis/trans-1,2-dichloroethylenes that are present in the product stream from step (a) are hydrogenated with hydrogen to form ethylene dichloride (1,2-dichloroethane), which optionally is recycled, at least in part, to the reactor in step (a).

17. The process of claim 9, wherein the oxygen is supplied as air.

18. The process of claim 17, wherein the oxygen in the feed is present at greater than three times the volumetric concentration of C2 hydrocarbon(s) (ethane and optional ethylene) in the feed.

19. The process of claim 17, wherein greater than about 95 mole percent of the C2 hydrocarbon fed is converted in step (a), such that essentially no recoverable C2 hydrocarbon is present in the product stream of step (a), and there is no need to for a C2 hydrocarbon recycle.

20. A method of manufacturing vinyl chloride monomer, comprising the steps of:
generating a first reactor effluent stream by catalytically reacting together ethane, ethylene, or a mixture thereof with an oxygen source and at least one chlorine source of hydrogen chloride, chlorine, or a saturated chlorohydrocarbon, wherein if both ethane and ethylene are present, then the molar ratio of said ethane to said ethylene is between 0.02/1 and 50/1, under fuel-lean process conditions sufficient to convert greater than about 95 mole percent of the C2 hydrocarbons fed;
cooling and condensing said first reactor effluent stream to provide a raw product stream having a first portion of said hydrogen chloride and a raw cooled hydrogen chloride stream having a second portion of said hydrogen chloride;
separating said raw product stream into a vinyl chloride monomer product stream and into a lights stream having said first portion of said hydrogen chloride;
recovering said second portion of said hydrogen chloride from said raw cooled hydrogen chloride stream;
and recycling said second portion of said hydrogen chloride to said reactor wherein ethane, ethylene, or a mixture thereof is combined with said oxygen source and said chlorine source.

21. A method of manufacturing vinyl chloride, comprising the steps of:
generating a first reactor effluent stream by catalytically reacting together ethane, optionally ethylene, an oxygen source, and at least one chlorine source of hydrogen chloride, chlorine, or a saturated chlorohydrocarbon, wherein if ethylene is present, then the molar ratio of said ethane to said ethylene is between 0.02/1 and 50/1;
cooling and condensing said first reactor effluent stream to provide a raw product stream having a first portion of said hydrogen chloride and a raw cooled hydrogen chloride stream having a second portion of said hydrogen chloride;
separating said raw product stream into a vinyl chloride monomer product stream and into a lights stream having said first portion of said hydrogen chloride;
catalytically reacting essentially all of said first portion of hydrogen chloride in said lights stream to provide a second reactor effluent essentially devoid of hydrogen chloride; and
recycling said second reactor effluent to catalytically react together with said ethane, said optional ethylene, said oxygen source, and said chlorine source in said generating step.
